(19) United States
(12) Patent Application Publication
(10) Pub. No.: US 2006/0141549 A1
(43) Pub. Date: Jun. 29, 2006

(54) Cell-based kinase assay

(76) Inventors: Sudipta Mahajan, Framingham, MA (US); Thomas Hoock, Westford, MA (US)

Correspondence Address: VERTEX PHARMACEUTICALS INC., 130 WAVERLY STREET, CAMBRIDGE, MA, 02139-4242, US

(21) Appl. No.: 11/195,550

(22) Filed: Aug. 2, 2005

Related U.S. Application Data
(60) Provisional application No. 60/598,294, filed on Aug. 3, 2004.

(51) Int. Cl. C12Q 1/48; C12Q 1/42

(52) U.S. Cl. 435/15; 435/21

(57) ABSTRACT

The present invention relates to improved systems and strategies for the investigation of kinase activity in cells. More specifically, cell-based assay methods are provided that allow the phosphorylating activity of a kinase to be determined inside a cell. The invention also provides cell-based screening assays for identifying compounds that have the ability to modulate the phosphorylating activity of protein kinases. Modulators of kinase activity identified by the screening methods are also described, as are pharmaceutical compositions comprising these modulators and methods of using them for inhibiting or enhancing cellular responses triggered by kinase-mediated events.

This application claims the benefit of priority from United States Provisional Application 60/598,294, filed Aug. 3, 2004, which is hereby incorporated by reference in its entirety.

### BACKGROUND OF THE INVENTION

While the cost of developing a new drug continues to increase and was reported to have reached approximately $800 million in 2000 (C. McNicholas and M. Duggan, Technology, 2002, 1: 46-50), the proportion of drugs progressing through the pipeline is still low. In order to improve their research and development productivity, companies in the pharmaceutical and biotechnology industry are more and more frequently adopting cell-based assays in the early phases of the drug discovery process. The use of cell-based assays is expected to reduce the late-stage failure rates of compounds in the pipeline by allowing improved, early selection of drug candidates with higher probability of success in pre-clinical and clinical trials (O. E. Beeske and S. Goldbard, Drug Discov. Today, 2002, 7: S131-S135).

Cell-based assays have remarkable advantages over biochemical assays which are generally performed under conditions that only marginally reproduce the context of a live cell. Since it remains difficult to assess the in vivo activity and specificity of a molecule based on its in vitro behavior, biochemical assays are likely to have only marginal biological relevance. In contrast, cell-based assays offer the opportunity to study the effects of a candidate compound on a drug target under conditions that more closely mimic the actual physiological situation. Furthermore, carrying out screening assays in cells also allows candidate compounds to be evaluated for cell permeability and toxicity. The availability of these important factors (which are not addressed in biochemical assays) can save valuable time and costs in the development of new drugs. In addition, cell-based assays do not require isolation and purification of the drug target (typically a target protein), which further reduces investment of time and resources. This latter advantage is particularly interesting considering the increasing number of proteins derived from genomics and proteomics that can be targeted for potential drug treatment. This is especially true in the case of protein kinases, which are considered as a major class of therapeutic targets.

Protein kinases constitute a large family of structurally related enzymes that are responsible for the control of a wide variety of cellular processes, including transcription and translation of genes, cell cycle regulation, cell growth, cell metabolism, apoptosis and differentiation (see, for example, G. Hardie and S. Hanks, “The Protein Kinase Facts Book, I and II”, 1995, Academic Press: San Diego, Calif.; T. Hunter, Cell, 1995, 80: 225-236; and M. Karin, Curr. Opin. Cell Biol. 1991, 3: 467-473). Kinases regulate these cellular processes by catalyzing the phosphorylation of amino acid residues of certain proteins. Protein phosphorylation generally occurs in response to different stimuli such as environmental or nutritional stresses, cell cycle checkpoints and extracellular signals (e.g., growth and differentiation factors, hormones and neurotransmitters). These stimuli act as molecular switches by inducing protein kinases to activate a particular metabolic enzyme, regulatory protein, cell-surface receptor, ion channel, ion pump, cytoskeletal protein or transcription factor. Reversible protein phosphorylation (i.e., phosphorylation by kinases and de-phosphorylation by phosphatases) controls and regulates most activities of eukaryotic cells and plays a critical role in an organism's maintenance and adaptation.

Abnormal expression and aberrant control of the enzymatic activity of kinases have been implicated in a large number of disease conditions including cancer (P. Dirks, Neurosurgery, 1997, 40: 1000-1013; V. Boudny and J. Kovarik, Neoplasma, 2002, 49: 349-355); neurodegenerative disorders such as Alzheimer's disease (K. Imahori et al., J. Biochem. 1997, 121: 179-188) and Parkinson's disease (J. Peng and J. K. Andersen, IUBMB Life, 2003, 55: 267-271; S. J. Harper and N. Wilkie, Expert Opin. Ther. Targets, 2003, 7: 187-200); rheumatoid arthritis (M. Piecyk and P. Anderson, Best Pract. Res. Clin. Rheumatol. 2001, 15: 789-803; D. Hammaker et al., Ann. Rheumatol. Dis. 2003, 62(Suppl): 86-89); inflammation and infection (J. Han et al., Nature, 1997, 386: 296-299); atherosclerosis (M. Boehm and E. G. Nabel, Prog. Cell Cycle Res. 2003, 5: 19-30); and diabetes (N. Alto et al., Diabetes, 2002, 51(Suppl): S385-388; F. B. Stenz and A. E. Kitabchi, Curr. Drug Targets, 2003, 4: 493-503). Since dysfunction in protein phosphorylation processes can have serious consequences for cellular regulatory mechanisms, protein kinases are attractive targets for drug discovery.

Most kinase activity studies have traditionally been performed using biochemical assays based on purified enzymes produced as recombinant proteins from insect or mammalian cells in culture. Although these assays lack the physiological context of the cell, they have been widely used and adapted to high-throughput drug screening. Cell-based methods that monitor kinase activity, for example in the presence of a potential drug candidate, have been developed that rely on the incorporation of ³²P into cells. Following ³²P incorporation and incubation in the presence of a drug candidate, the cells are lysed and the substrate protein is isolated and purified to determine its relative degree of phosphorylation by measuring the amount of ³²P incorporated. Such cell-based assays are labor intensive and only poorly sensitive, and have the disadvantage of requiring high numbers of cells and high levels of radioactivity. Other cell-based assays for the study of kinase activity use radiolabeled phosphorylation-specific antibodies (i.e., antibodies that can distinguish between phosphorylated and non-phosphorylated proteins). In these assays, the phosphorylated substrate protein is detected and quantified by immunoprecipitation, gel electrophoresis or Western blotting after lysis of the cells. Although these assays generally require lower levels of radioactivity than ³²P-based methods, they are equally labor intensive, time consuming and complex to automate.

More recently, non-radioactive cell-based methods have emerged that use an ELISA (i.e., enzyme-linked immunosorbent assay) approach to measure the activation of specific kinase signaling pathways. These kinase assays, which employ phosphorylation-specific antibodies, have been demonstrated to be suitable for high-throughput drug screening (H. H. Versteeg et al., Biochem. J. 2000, 350: 717-720). However, like most other currently available cell-based methods for measuring protein phosphorylation, these assays require cell lysis, which implies that any corresponding read-outs will represent an average for protein activation states across the entire cell population(s) studied. Such averaging does not allow potential differences or variations between individual cells to be detected and therefore may mask significant biological information on the distribution of protein activation within a cell population.

Clearly, improved methods are still needed for the qualitative and quantitative assessment of kinase activity, as well as for the identification of modulators of such kinase activity under conditions that most closely mimic the actual in vivo situation. In particular, cell-based assays that are simple, rapid, sensitive and adaptable to high-throughput screening, that provide information about individual cells within a cell population, and that allow the potential therapeutic value of candidate compounds to be evaluated in the early phases of the drug discovery and development process are highly desirable.

### SUMMARY OF THE INVENTION

The present invention relates to improved strategies for the investigation of kinase activity in cells. In particular, systems are provided that have the advantage of performing a multi-parametric cell-by-cell analysis for a large number of cell samples in a short period of time. More specifically, the present invention is directed to cell-based assay methods that allow the phosphorylating activity of a kinase to be determined when the kinase is constitutively active or when it is activated in the presence of an extracellular stimulus. The inventive methods may be used for screening candidate compounds and identifying those compounds that modulate kinase activity in cells. The methods of the invention, which include using a Flow Cytometry Plate Reader, are simple and sensitive high-throughput assays that can easily be applied to study the phosphorylating activity of a wide variety of protein kinases. Furthermore, in addition to requiring only small amounts of cells and reagents, the inventive methods also have the advantage of providing substantially more information in less time than other conventional kinase assays. This ultimately results in faster identification and more relevant evaluation of promising drug candidates.

In one aspect, the present invention is directed to methods for measuring the phosphorylating activity of an enzyme in cells, wherein the enzyme is a protein kinase catalyzing the phosphorylation of a substrate molecule involved in a signaling pathway. The inventive methods comprise steps of: providing cells in a plurality of wells of a multi-well assay plate; exposing cells to a fluorescently-detectable selective probe such that the probe binds to the phosphorylated substrate; measuring the amount of probe bound to the phosphorylated substrate using a Flow Cytometry Plate Reader; and based on the amount of bound probe, determining the phosphorylating activity of the kinase.

The inventive methods may be used to study the phosphorylating activity of a wide variety of protein kinases including constitutively active kinases and non-constitutively active kinases; transmembrane (i.e., receptor) kinases and intracellular (i.e., non-receptor) kinases; tyrosine kinases, serine/threonine kinases, histidine kinases and dual-specificity kinases.

Cells to be used in the methods of the invention may be primary cells, secondary cells or immortalized cells, of any cell type and origin. Preferably, cells are of mammalian origin, including human. In certain embodiments, cells are of different cell types. In other embodiments, cells are from a substantially homogeneous population of cells. The methods of the invention allow analysis of large numbers of cell samples contained, for example, in 42-, 96-, 384-, or 1536-well assay plates. In those embodiments where the multi-well assay plate is a 96-well plate, between about 1×10⁴ and about 50×10⁴ cells are preferably present per well.

When the phosphorylating activity of a non-constitutively active protein kinase is under investigation, the methods of the invention may comprise additional steps, such as starving the cells prior to exposing them to a kinase activator so that activation of the protein kinase takes place and results in phosphorylation of the substrate molecule. The kinase activator may be an environmental stress signal (such as osmotic shock, heat shock, hypoxia, and UV radiation), a chemical stress signal (such as oxidative stress, human carcinogens, and environmental pollutants), a biochemical stimulus (such as growth factors, cytokines, growth hormones, and neurotransmitters), or any combinations of these stimuli.

In certain preferred embodiments, the inventive methods further comprise fixing and permeabilizing the cells, and optionally storing the assay plate for a certain period of time, before exposing the cells to a fluorescently-detectable selective probe.

In certain embodiments, exposing the cells to a fluorescently-detectable selective probe includes adding to the cells a phospho-specific antibody comprising a fluorescent label. In other embodiments, exposing the cells to a fluorescently-detectable selective probe includes adding to the cells a phospho-specific antibody and a secondary antibody, which specifically binds to the phospho-specific antibody and comprises a fluorescent label. The phospho-specific antibody may be a monoclonal or polyclonal antibody. Preferably, the phospho-specific antibody recognizes and binds to at least one phosphorylated residue of the phosphorylated substrate, for example, a phosphorylated tyrosine, a phosphorylated serine, a phosphorylated threonine, or a phosphorylated histidine residue. The substrate molecule undergoing phosphorylation may be, for example, a downstream protein kinase, a gene regulatory protein, a cytoskeletal protein or a metabolic enzyme.

In the methods of the present invention, determining the phosphorylating activity of a given kinase includes measuring the amount of fluorescently-detectable selective probe bound to the phosphorylated substrate using a Flow Cytometry Plate Reader. In preferred embodiments, measuring the amount of selective probe bound to the phosphorylated substrate comprises measuring the intensity of a fluorescence signal from one or more cells in each well of the multi-well assay plate. Preferably, the signal is generated by a fluorescent label. The fluorescent label may comprise a quantum dot (i.e., a fluorescent inorganic semiconductor nanocrystal) or a fluorescent dye, such as, for example, Texas red, fluorescein isothiocyanate (FITC), phycoerythrin (PE), rhodamine, fluorescein, carbocyanine, Cy-3™, Cy-5™, merocyanine, styryl dye, oxonol dye, BODIPY dye, and the like.

In certain embodiments, the methods of the invention further comprise measuring light scatter from one or more cells in each one of the plurality of wells containing cells using the Flow Cytometry Plate Reader. Light scatter measurements may be used to get insight into characteristics such as cell shape, cell size and cytoplasmic granularity.

In another aspect, the present invention is directed to methods for identifying candidate compounds that have the ability to modulate the phosphorylating activity of an enzyme in cells, wherein the enzyme is a protein kinase catalyzing the phosphorylation of a substrate molecule involved in a signaling pathway. The inventive methods comprise steps of: providing cells in a plurality of wells of a multi-well assay plate; incubating cells in some wells of the assay plate with a candidate compound under conditions and for a time sufficient to allow equilibration, thus obtaining test cells; incubating cells in other wells of the assay plate under the same conditions and for the same time absent the candidate compound, thus obtaining control cells; exposing the test and control cells to a fluorescently-detectable selective probe such that the selective probe binds to the phosphorylated substrate; measuring the amount of selective probe bound to the phosphorylated substrate in the test and control cells using a Flow Cytometry Plate Reader; comparing the amount of bound probe in the test and control cells; and determining that the candidate compound modulates the phosphorylating activity of the protein kinase studied if the amount of bound probe in the test cells is less than or greater than the amount of bound probe in the control cells.

The cell systems, protein kinases, kinase activators, phospho-specific antibodies, and fluorescent labels described above are also suitable for use in the practice of the screening methods of the invention. Furthermore, steps of starving the cells and exposing them to a kinase activator in the case of non-constitutively active kinases; of fixing and permeabilizing the cells; of storing the assay plate for a certain period of time before staining and analysis; and of measuring light scatter from each analyzed cell using a Flow Cytometry Plate Reader may also be carried out in the inventive screening methods.

The screening methods of the invention may be used to identify candidate compounds that are inhibitors or stimulators of the phosphorylating activity of a given kinase. The inventive methods may be used to test individual candidate compounds for their ability to modulate the phosphorylating activity of a kinase. Alternatively, the inventive methods may be used to screen collections or libraries of candidate compounds and identify modulators of kinase activity. For example, the inventive methods may be used to test small molecules or to screen libraries of small molecules.

In certain embodiments, candidate compounds are tested at varying concentrations, for example, between about 10 pM and about 100 μM. Screening candidate compounds at varying concentrations allows IC₅₀ values to be determined for these compounds. In other embodiments, the inventive methods further comprise the use of positive and/or negative control compounds and comparison of the modulating effects of candidate compounds with the modulating effects of the positive and/or negative control compounds.

In another aspect, the present invention is directed to compounds that are/have been identified by a screening method described herein as modulators (i.e., inhibitors or stimulators) of the phosphorylating activity of a given kinase. Also provided are pharmaceutical compositions comprising at least one physiologically acceptable carrier and an effective amount of at least one modulator.

In still another aspect, the present invention is directed to a method for inhibiting or enhancing a kinase activity inside a cell. The method comprises the step of contacting the cell with an effective amount of a compound identified by an inventive screening method as an inhibitor of kinase activity or as a stimulator of kinase activity.

In yet another aspect, the present invention is directed to a method for inhibiting or enhancing a kinase activity in a system, wherein the kinase activity is associated with abnormal cellular responses. The method comprises a step of contacting the system with an effective amount of a compound identified as an inhibitor of kinase activity or as a stimulator of kinase activity. The system may be a cell, a biological fluid, a biological tissue or a mammal, for example, an animal model for a human disease or pathophysiological condition associated with abnormal cellular responses resulting from kinase-mediated events.

### BRIEF DESCRIPTION OF THE DRAWINGS

FIG. 1 shows a histogram exhibiting the fluorescent intensity at different drug concentrations when HT-2 cells are pre-incubated with a candidate compound for 1 hour, stimulated with IL-2 for 20 minutes, and stained for the phospho STAT-5 PE antibody as described in Example. As the drug concentration increases the percentage of cells staining positive for Phospho STAT-5 PE decreases.

FIG. 2 shows a 4-parameter curve of percentage of HT-2 cells positive for Phospho STAT-5 PE as a function of drug concentration. This graph is used to calculate the IC₅₀ value based on maximum signal in cells that were not incubated in the presence of the candidate compound.

FIG. 3 shows a histogram exhibiting the fluorescent intensity at different drug concentrations when TF-1 cells are pre-incubated with a candidate compound for 1 hour, stimulated with GM-CSF for 15 minutes, and stained for the phospho STAT-5 PE antibody as described in Example 2. As the drug concentration increases the percentage of cells staining positive for Phospho STAT-5 PE decreases.

FIG. 4 shows a 4-parameter curve of percentage of TF-1 cells positive for Phospho STAT-5 PE as a function of drug concentration. This graph is used to calculate the IC₅₀ value based on maximum signal in cells that were not incubated in the presence of the candidate compound.

### Definitions

Throughout the specification, several terms are employed that are defined in the following paragraphs.

The terms “kinase” and “protein kinase” are used herein interchangeably. They refer to an enzyme that catalyzes the transfer of a phosphate group from a nucleoside triphosphate to certain amino acid residues of another molecule (herein called “substrate” or “kinase substrate”) that is involved in a signaling pathway. The phosphate group may be transferred, for example, from an ATP (adenosine triphosphate) or GTP (guanosine triphosphate) molecule. Kinases may be transmembrane (i.e., receptor) or intracellular (i.e., non-receptor) proteins. Eukaryotic protein kinases are characterized by the sequence of a contiguous stretch of approximately 250 amino acids that constitutes the catalytic (kinase) domain. Although no residue in this region is absolutely conserved in all family members, there are a number of conserved regions in the catalytic domain that can be used to determine that a particular protein belongs in the kinase family. For example, in the N-terminal extremity of the catalytic domain, there is a glycine-rich stretch of residues in the vicinity of a lysine residue, which has been shown to be involved in ATP binding. In the central part of the catalytic domain, there is a conserved aspartic acid residue which is important for the catalytic activity of the enzyme. The pattern of residue conservation seen within this core of 250 amino acids is thought to be due to selective evolutionary pressure to preserve the major function of this gene family, i.e., the catalysis of phosphate transfer from ATP to substrate molecules. Specific examples of protein kinases whose phosphorylating activity may be assessed by the methods of the invention are listed in the Detailed Description.

The terms “tyrosine kinase”, “serine/threonine kinase”, and “histidine kinase” are used herein to refer to an enzyme that specifically catalyzes the phosphorylation of substrate molecules at one or more tyrosine residues, serine/threonine residues and histidine residues, respectively. The term “dual-specificity kinase” refers to an enzyme that catalyzes the phosphorylation of serine/threonine residues and/or tyrosine residues of substrate molecules.

The terms “phosphorylating activity” and “kinase activity” are used herein interchangeably. They refer to the ability of a kinase to catalyze the phosphorylation of certain amino acid residues of a substrate molecule. The terms “tyrosine kinase activity”, “serine/threonine kinase activity”, and “histidine kinase activity” are used to refer to the ability of a protein kinase to specifically catalyze the phosphorylation of tyrosine residues, serine/threonine residues, and histidine residues, respectively.

The terms “substrate” and “kinase substrate” are used herein interchangeably. They refer to a molecule involved in one or more signaling pathways, which can become phosphorylated through the action of a kinase, and whose phosphorylation ultimately results in the modification of one or more cellular responses. Cellular responses may be related, for example, to cell growth, migration, differentiation, secretion of hormones, activation of transcription factors, muscle contraction, glucose metabolism, control of protein synthesis, and/or regulation of the cell cycle. Exemplary substrates include, but are not limited to, metabolic enzymes, gene regulatory proteins, cytoskeletal proteins or other protein kinases (e.g., downstream kinases that participate in the same signaling pathway than the kinase whose phosphorylating activity is under investigation in the assay).

The term “kinase activator”, as used herein, refers to any extracellular or other type of stimulus that triggers activation of a kinase, which in turn induces phosphorylation of a substrate molecule. Examples of kinase activators include environmental stress signals (such as osmotic shock, heat shock, hypoxia, and UV radiation), chemical stress signals (such as oxidative stress, human carcinogens, and environmental pollutants), and biochemical stimuli (such as growth factors, cytokines, growth hormones, and neurotransmitters). Biochemical stimuli are generally molecules naturally secreted by cells that affect the function of other cells. Specific examples of biochemical stimuli that can be used as kinase activators in the assays of the invention are given in the Detailed Description.

The term “constitutively active” when applied to a protein kinase refers to a kinase that has the ability to catalyze substrate phosphorylation in the absence of a kinase activator. Constitutively active kinases may be tyrosine kinases, serine/threonine kinases, histidine kinases or dual-specificity kinases. Constitutively active kinases may be endogenously expressed in the cells studied in the assays or, alternatively, cells may be transformed to express a constitutively actively kinase.

As used herein, the term “substantially homogeneous population” when applied to cells, refers to a population of cells, wherein at least about 80%, and preferably about 90% of the cells in the population are of the same cell type. Examples of cell types include, but are not limited to, platelets, lymphocytes, T-cells, B-cells, natural killer cells, endothelial cells, tumor cells, epithelial cells, granulocytes, monocytes, mast cells, neurocytes, and the like.

The term “fluorescently-detectable” when applied to a probe is used to specify that the probe can be visualized by fluorescence. To be fluorescently-detectable, a probe may be conjugated or linked to a fluorescent label (for example, the probe may be a phospho-specific antibody comprising a fluorescent molecule), or may be specifically recognized by a secondary probe that is conjugated or linked to a fluorescent label (for example, the probe may be a phospho-specific antibody that is specifically recognized by a secondary antibody comprising a fluorescent molecule).

The terms “fluorophore” and “fluorochrome” are used herein interchangeably. They refer to a molecule which, in solution and upon excitation with light of appropriate wavelength, emits light back. The term “fluorescent label” refers to a fluorescent molecule that can be covalently attached to a probe (for example, an antibody) such that this probe becomes detectable by fluorescence. Numerous fluorescent labels of a wide variety of structures and characteristics are suitable for use in the practice of this invention. Preferred fluorophores are photostable (i.e., they do not undergo significant degradation upon light excitation within the time necessary to perform the analysis). Suitable fluorophores include, but are not limited to, quantum dots (i.e., fluorescent inorganic semiconductor nanocrystals) and fluorescent dyes such as, for example, fluorescein, rhodamine, cyanine, carbocyanine, allophycocyanine, phycoerythrin, umbelliferone, and derivatives, analogues and combinations thereof.

As used herein, the term “selective probe” refers to any molecule, compound, agent or moiety that exhibits a specific affinity for a phosphorylated substrate under the conditions of a binding assay. Selective probes recognize and bind to particular phosphorylated substrate molecules. The term “recognize(s) and bind(s) to” is meant to include detectable biochemical interactions between the probe and the phosphorylated substrate, such as protein-protein, protein-nucleic acid, nucleic acid-nucleic acid, protein-organic or inorganic molecule, and nucleic acid-organic or inorganic molecule interactions. A probe is selective if it recognizes and binds to one or more target substrates while excluding non-target molecules within a given sample. Selective probes suitable for use in the methods of the invention include, but are not limited to, biomolecules such as proteins, phospholipids, and DNA hybridizing probes. Preferred selective probes are phospho-specific antibodies.

The term “antibody”, as used herein, refers to any immunoglobulin, including antibodies (i.e., intact immunoglobulin molecules) and fragments thereof (i.e., active portions of immunoglobulin molecules), that binds to a specific epitope. The term encompasses monoclonal antibodies and antibody compositions with polyepitopic specificity (i.e., polyclonal antibodies).

The term “phospho-specific antibody” (or “phosphorylation-specific antibody”) refers to an antibody which selectively recognizes and binds to phosphorylated residues of a substrate molecule. Preferred phospho-specific antibodies selectively recognize and bind to one type of phosphorylated amino acid residue. For example, an anti-phosphotyrosine antibody binds selectively to phosphorylated tyrosine residues of a kinase substrate. Phospho-specific antibodies and their methods of preparation are known in the art. Phospho-specific antibodies are also commercially available, for example, from New England Biolabs, Inc. (Beverly, Mass.), BD Biosciences/Pharmingen (San Diego, Calif.), Sigma-Genosys (the Woodlands, Tex.), and Upstate Biologicals, Inc. (Lake Placid, N.Y.).

The term “Flow Cytometry Plate Reader”, as used herein, refers to an instrument that can perform a flow cytometric analysis of samples of cells in suspension, which are, for example, contained in wells of an assay plate. In particular, a Flow Cytometry Plate Reader can perform a multi-parametric cell-by-cell analysis for a large number of cell samples in a short period of time. Preferably, a Flow Cytometry Plate Reader is manufactured with the ability to measure more than one different detectable label simultaneously, as well as light scatter from each analyzed cell. Preferred Flow Cytometry Plate Readers for use in the methods of the invention are similar or identical to those commercially available from Guava Technologies (Hayward, Calif.), in particular the Guava PCA-96 system, or from BD Biosciences (San Jose, Calif.), in particular the BD FACSArray™ Bioanalyzer System.

The term “candidate compound” refers to any naturally occurring or non-naturally occurring molecule, such as a biological macromolecule (e.g., nucleic acid, polypeptide or protein), organic or inorganic molecule, or an extract made from biological materials such as bacteria, plants, fungi, or animal (particularly mammalian, including human) cells or tissues to be tested for an activity of interest. In the screening methods of the invention, candidate compounds are evaluated for their ability to modulate the phosphorylating activity of a given kinase inside a cell.

The term “small molecule”, as used herein, refers to any natural or synthetic organic or inorganic compound or factor with a low molecular weight. Preferred small molecules have molecular weights of more than 50 Daltons and less than 2,500 Daltons. More preferably, small molecules have molecular weights of less than 600-700 Daltons. Even more preferably, small molecules have molecular weights of less than 350 Daltons.

As used herein, the term “modulation of phosphorylating activity or kinase activity” refers to the ability of a candidate compound to enhance (e.g., stimulate or increase) or inhibit (e.g., fully suppress or partially decrease) the ability of a protein kinase to catalyze the transfer of a phosphate group from a nucleoside triphosphate to certain amino acid residues of a substrate molecule. By “inhibition” is meant that the level of phosphorylation of the substrate is reduced at least 50% after incubation in the presence of a candidate compound tested in the assay. Preferably, the level of phosphorylation of the substrate is reduced at least 90% by the candidate compound. More preferably, the level of phosphorylation of the substrate is reduced at least 95% by the candidate compound. By “enhancement” or “stimulation” is meant that the level of phosphorylation of the substrate is increased at least 2 to 3 fold after incubation in the presence of a candidate compound tested in the assay. Preferably, the level of phosphorylation of the substrate is increased at least 5 fold by the candidate compound. More preferably, the level of phosphorylation of the substrate is increased at least 10 fold by the candidate compound. A candidate compound that induces such an inhibition or enhancement of the level of phosphorylation of a substrate molecule in a kinase assay of the invention is “identified” as a modulator of the phosphorylating activity of the kinase. Thus, a “modulator of phosphorylating activity” is a compound that is/has been identified by a screening method of the invention as inhibiting/suppressing or enhancing/stimulating the phosphorylating activity of a given kinase.

A “pharmaceutical composition” is herein defined as comprising a physiologically acceptable carrier and an effective amount of at least one inventive modulator of kinase activity.

As used herein, the term “effective amount” refers to any amount of a modulator of kinase activity, or pharmaceutical composition thereof, that is sufficient to achieve an intended purpose. For example, the intended purpose may be: to inhibit or enhance the phosphorylating activity of a kinase when the kinase is constitutively active or when the kinase is stimulated by a kinase activator inside a cell; to inhibit or enhance cellular response(s) resulting from kinase-mediated events; and/or to prevent or treat a disease or pathophysiological condition associated with abnormal cellular responses resulting from kinase-mediated events.

As used herein, the term “physiologically acceptable carrier” refers to a carrier medium which does not interfere with the effectiveness of the biological activity of the active ingredients and which is not excessively toxic to the host at the concentrations at which it is administered. The term includes solvents, dispersion media, coatings, antibacterial and antifungal agents, isotonic agents, absorption delaying agents, and the like. The use of such media and agents for pharmaceutically active substances is well known in the art (see, for example, Remington's Pharmaceutical Sciences, E. W. Martin, 18^{th} Ed., 1990, Mack Publishing Co., Easton, Pa., which is incorporated herein by reference in its entirety).

As used herein, the term “system” refers to an in vitro, in vivo or ex vivo biological entity such as a cell, a biological fluid, a biological tissue or an animal. A system may, for example, originate from a live individual (e.g., it may be obtained by biopsy or by drawing blood) or from a deceased individual (e.g., it may be obtained at autopsy). The individual may be a human or another mammal. For example, the individual may be an animal model for a human disease or medical condition associated with abnormal cellular responses associated with kinase-mediated events.

### DETAILED DESCRIPTION OF CERTAIN PREFERRED EMBODIMENTS

Improved systems and strategies for the investigation of kinase activity and for the identification of modulators of phosphorylating activity are described herein. In particular, cell-based high-throughput methods are provided that involve determination of the level of kinase activity by measuring the amount of phosphorylated substrate inside a single cell using a Flow Cytometry Plate Reader. The inventive methods are multi-parametric, rapid and quantitative, and have the advantage, among others, of providing an individual cell-based mode of analysis rather than a bulk population assessment.

I. Determination of the Phosphorylating Activity of a Kinase Inside a Cell

In one aspect, the invention provides assays for determining the phosphorylating activity of a kinase inside a cell.

More specifically, a method is provided for measuring the phosphorylating activity of an enzyme, wherein the enzyme is a protein kinase catalyzing the phosphorylation of a substrate molecule that is involved in a signaling pathway. The inventive method comprises steps of: providing cells in a plurality of wells of a multi-well assay plate; exposing the cells to a fluorescently-detectable selective probe such that the probe binds to the phosphorylated substrate; measuring the amount of probe bound to the phosphorylated substrate using a Flow Cytometry Plate Reader, and, based on the amount of probe bound to the phosphorylated substrate, determining the phosphorylating activity of the kinase. The kinase, whose phosphorylating activity is studied by the inventive method, may be constitutively active or may be stimulated by exposing the cells to a kinase activator such that activation of the kinase results in phosphorylation of the substrate molecule.

Cells, Culture and Preparation

The methods provided by the present invention are cell-based assays. As already mentioned above, cell-based assays have key advantages over biochemical assays (see, for example, J. R. Zysk and W. R. Baumbach, Comb. Chem. High Throughput Screen, 1998, 1: 171-183; J. N. Weinstein and J. K. Buolamwini, Curr. Pharm. Des. 2000, 6: 473483; D. L. Taylor et al., Curr. Opin. Biotechnol. 2001, 12: 75-81; and J. H. Price et al., J. Cell Biochem. Suppl. 2002, 39: 194-210). Biochemical target binding assays do not address drug efficacy and toxicity in a relevant biological context. Screening in cells tests not only the effects of compounds on a drug target in a biologically relevant environment but also simultaneously evaluates candidate compounds for cell permeability, toxicity, and other factors not addressed in biochemical assays. Since such parameters are assessed by the cell-based assay itself, it is not necessary to design and perform extensive additional toxicity controls, cell permeability analyses and stability experiments, which generally follow traditional in vitro biochemical screening approaches. This allows cell-based assay development and optimization to proceed rapidly, accelerating the early phases of target validation and lead discovery.

The assay and screening methods of the present invention may be carried out using any cell types that can be grown in standard tissue culture plastic ware. Such cell types include all normal and transformed cells derived from any recognized sources, for example, mammalian, plant, bacterial, viral or fungal. However, preferably, cells are of mammalian (human or animal, such as rodent or simian) origin. More preferably, cells are of human origin. Mammalian cells may be of any organ or tissue origin (e.g., brain, liver, lung, heart, kidney, skin, muscle, bone, bone marrow or blood) and of any cell types. Suitable cell types include, but are not limited to, basal cells, epithelial cells, platelets, lymphocytes, T-cells, B-cells, natural killer cells, reticulocytes, granulocytes, monocytes, mast cells, neurocytes, neuroblasts, cytomegalic cells, dendritic cells, macrophages, blastomeres, endothelial cells, tumor cells, interstitial cells, Kupffer cells, Langerhans cells, littoral cells, tissue cells such as muscle cells and adipose cells, enucleated cells, and the like.

Cells to be used in the practice of the methods of the present invention may be primary cells, secondary cells or immortalized cells (i.e., established cell lines). They may be prepared by techniques well known in the art (for example, cells may be obtained by drawing blood from a patient or healthy donor) or purchased from immunological and microbiological commercial resources (for example, from the American Type Culture Collection, Manassas, Va.). Alternatively or additionally, cells may be genetically engineered to contain, for example, a gene of interest such as a gene expressing a growth factor or a receptor.

In certain embodiments, the cells used in the inventive screening methods are of more than one cell type. In other embodiments, the cells are of a single cell type. Preferably, cells are from a substantially homogeneous population of cells, wherein at least about 80%, and preferably at least about 90% of the cells in the population are of the same cell type. Cells to be used in the methods of the invention may originate from different individuals of the same species. However, preferably, cells originate from a single individual.

Selection of a particular cell type and/or cell line to develop a kinase assay according to the present invention will be governed by several factors such as the nature of the protein kinase whose phosphorylating activity is to be studied and the intended purpose of the assay. For example, an assay developed for primary drug screening (i.e., first round(s) of screening) may preferably be performed using established cell lines, which are commercially available and usually relatively easy to grow, while a kinase assay to be used later in the drug development process may preferably be performed using primary or secondary cells, which are often more difficult to obtain, maintain, and/or to grow than immortalized cells but which represent better experimental models for in vivo situations. Primary and secondary cells that can be used in the inventive screening methods, include, but are not limited to, peripheral blood mononuclear cells, T-cells, bone-marrow mononuclear cells, retinoblasts, and the like.

Selection of a particular cell line to develop a kinase assay according to the present invention can readily be performed by one of ordinary skill in the art. For instance, in Example 1, an Interleukin-2 (IL-2) dependent murine T lymphocyte cell line (HT-2 cells) was used to study the phosphorylating activity of Janus kinase 3 (JAK3) on Signal Transducer and Activator of Transcription protein 5 (STAT-5). In Example 2, an erythroleukemia cell line (TF-1 cells) known to be dependent on the cytokine Granulocyte Macrophage-Colony Stimulating Factor (GM-CSF) for growth was used to study the phosphorylating activity of Janus kinase 2 (JAK2) on STAT-5.

Cells to be used in the inventive assays may be cultured according to standard cell culture techniques. For example, cells are often grown in a suitable vessel in a sterile environment at 37° C. in an incubator containing a humidified 95% air—5% CO₂ atmosphere. Vessels may contain stirred or stationary cultures. Various cell culture media may be used including media containing undefined biological fluids such as fetal calf serum, as well as media which are fully defined, such as 293 SFM serum free medium (Invitrogen Corp., Carlsbad, Calif.). Cell culture techniques are well known in the art and established protocols are available for the culture of diverse cell types (see, for example, R. I. Freshney, “Culture of Animal Cells: A Manual of Basic Technique”, 2^{nd} Edition, 1987, Alan R. Liss, Inc., which is incorporated herein by reference in its entirety).

If desired, cell viability can be determined, prior to the assay, for example, using standard techniques including histology, quantitative assessment with radioisotopes, visual observation using a light or scanning electron microscope or a fluorescent microscope. Alternatively, cell viability may be assessed by Fluorescence-Activated Cell Sorting (FACS).

In certain embodiments, the inventive methods comprise a step of starving the cells before exposing them to different reagents. Cell starvation may be particularly useful when the protein kinase of interest is not constitutively active. Starving interrupts the normal cycle of cellular growth and division, places the cells in a resting (inactivated) state, and brings the cells' phosphorylation level to a baseline. The starvation conditions and starvation period should preferably be selected to allow most cells of the sample (e.g., more than 80% of the cells; preferably more than 90% of the cells; more preferably more than 95% of the cells) to reach a resting state while avoiding cell deterioration or cell death. Synchronization of the cells into a resting state provides a population of cells that is substantially homogeneous in terms of activation.

Starving the cells may be performed by any suitable method, for example by culturing the cells in a medium without serum or growth supplements. In Example 1, HT-2 cells, which are dependent on IL-2 for their viability and proliferation, are starved by culturing them at 37° C. in a humidified incubator for 4 hours in the absence of the growth supplement, Rat T-STIM. In Example 2, TF-1 cells, which are dependent on GM-CSF for their growth, are starved by culturing them at 37° C. in a humidified incubator for 4 hours in the absence of GM-CSF.

Cell-based assays of the invention include providing cells into a plurality of (i.e., one or more) wells of a multi-well assay plate. Preferably, the assay plate is dimensioned and arranged for automated handling and/or analysis. Such assay plates are commercially available, for example, from Stratagene Corp. (La Jolla, Calif.) and Corning Inc. (Acton, Mass.) and include, for example, 48-well, 96-well, 384-well and 1536-well plates. The assay plate used by the Applicants in the experiments reported in Example 1 and Example 2, is a standard 96-V bottom well microtiter plate (86 mm by 129 mm).

The number of cells to be added to each well will depend on the size of the wells (i.e., the number of wells per plate). However, the number of cells to be added to each well should preferably be such that a significant number of cells (e.g., more than 2,000 or more than 5,000 cells per well) can be analyzed by the Flow Cytometry Plate Reader. For example, in the case of a 96-well assay plate, between about 1×10⁴ and about 50×10⁴ cells are preferably added to (or are present in) each well.

In certain methods of the invention, exposing cells to a reagent, contacting cells with a reagent, or incubating cells with a reagent comprises adding the reagent to a well containing cells and incubating the cells in the presence of the reagent in a suitable culture medium under conditions and for a period of time such that the intended role of the reagent is or can be achieved. More specifically, exposing cells to a kinase activator should be carried out under conditions that allow the (non-constitutively active) protein kinase of interest to be activated, thus leading to phosphorylation of the substrate molecule. Exposing cells to a fluorescently-detectable selective probe should preferably be carried out under conditions that allow the selective probe to specifically recognize and bind to the phosphorylated substrate. Exposing cells to a candidate compound to be tested for its effects on the phosphorylating activity of a given kinase should preferably be carried out under conditions that would allow a known modulator of such kinase activity to exert its inhibitory or enhancing effects. Such conditions are either well known in the art or may readily be determined, for example empirically, by one of ordinary skill in the art.

In certain embodiments, the assay and screening methods of the invention include fixing the cells. This step is performed to preserve or “freeze” a cell in a certain state, preferably so that an accurate representation of the structure of the cell is maintained. For example, it is often desirable to maintain the cell's original size and shape, to minimize loss of cellular materials, and/or to retain the reactivity and/or status of its intracellular constituents (for example, the cell's phosphorylation level). Cells may be fixed by any of a variety of suitable chemical and physical methods. Preferably, such a method is compatible with multi-well plate format assays. Methods of cell fixation typically rely on crosslinking and/or rapid dehydration agents, such as formaldehyde, paraformaldehyde, glutaraldehyde, acetic acid, methanol, ethanol, and acetone. Preferably, one or more fixing agents are added to cells contained in the well of an assay plate. Cells are preferably incubated in the presence of the fixing agent at a certain temperature (for example at room temperature, i.e., between 18° C. and 25° C.) and for a certain period of time (for example between 5 and 10 minutes). Excess fixing agent may be removed after centrifugation by aspiration of the supernatant.

In certain embodiments, the step of fixing the cells is followed by permeabilizing the cells. Permeabilization is performed to facilitate access to cellular cytoplasm or intracellular molecules, components or structures of a cell. In particular, permeabilization may allow an agent (such as a phospho-selective antibody) to enter into a cell and reach a concentration within the cell that is greater than that which would normally penetrate into the cell in the absence of such permeabilizing treatment.

Permeabilization of the cells may be performed by any suitable method (see, for example, C. A. Goncalves et al., Neurochem. Res. 2000, 25: 885-894). These methods include, but are not limited to, exposure to a detergent (such as CHAPS, cholic acid, deoxycholic acid, digitonin, n-dodecyl-β-D-maltoside, lauryl sulfate, glycodeoxycholic acid, n-lauroylsarcosine, saponin, and triton X-100) or to an organic alcohol (such as methanol and ethanol). Other permeabilizing methods comprise the use of certain peptides or toxins that render membranes permeable (see, for example, O. Aguilera et al., FEBS Lett. 1999, 462: 273-277; and A. Bussing et al., Cytometry, 1999, 37: 133-139). Preferably, in the kinase assays of the invention, permeabilization is performed by addition of an organic alcohol to the cells. Selection of an appropriate permeabilizing agent and optimization of the incubation conditions and time can easily be performed by one of ordinary skill in the art. As described in Examples 1 and 2, cells may be permeabilized in the presence of 90% methanol and incubated on ice for 30 minutes. Following this treatment, the assay plate may be stored at −20° C. for up to one month before being analyzed.

A flow cytometric analysis requires cells to be in suspension. Both adherent and non-adherent (i.e., suspension) cells may be used in the assays of the invention. However, when adherent cells are used, they need to undergo an additional treatment to allow detachment of the cells from their support in order to obtain a cell suspension. This can be achieved, for example, by trypsinization. Cell detachment may be performed at any stage of the kinase assay. Preferably, detachment of adherent cells is carried out before the step of staining.

Kinases and Kinase Activity

The assay and screening methods provided herein allow the level of phosphorylating activity of a given kinase to be assessed by measuring the amount of phosphorylated substrate.

Kinases regulate many different cell proliferation, differentiation, and signaling processes by effecting the transfer of a phosphate group from a nucleoside triphosphate to a substrate molecule involved in a signaling pathway. These phosphorylation events act as molecular on/off switches that can modulate or regulate the substrate's biological function. In the case of non-constitutively active kinases, phosphorylation of a substrate molecule results from kinase stimulation, which can occur in response to a variety of extracellular or other stimuli, such as environmental and chemical stress signals, cytokines, hormones and growth factors.

Kinases, which comprise the largest enzyme superfamily, vary widely in their selectivity and specificity of substrate molecules. Protein kinases can be divided into three main groups based on the amino acid sequence similarity or specificity for either tyrosine, serine/threonine or histidine residues. A small number of kinases have dual-specificity and phosphorylate both serine/threonine and tyrosine residues. Within the broad classification, kinases can be further sub-divided into families whose members share a higher degree of catalytic domain amino acid sequence identity and also have similar biochemical properties. Most protein kinase family members also share structural features outside the kinase domain that reflect their particular cellular roles. These include regulatory domains that control enzymatic activity or interaction with other proteins (S. K. Hanks et al., Science, 1988, 241: 42-52, which is incorporated herein by reference in its entirety).

Kinases whose phosphorylating activity can be assessed by the methods of the invention may be tyrosine, serine/threonine, histidine or dual-specificity kinases.

For example, screening methods of the invention may be developed that target a particular protein kinase of the tyrosine kinase family. Tyrosine kinases may occur as either transmembrane (i.e., receptor) or intracellular (i.e., non-receptor) proteins. Of the 90 tyrosine kinase genes identified in the human genome, 58 are receptor type (distributed in 20 subfamilies) and 32 are non-receptor type (distributed in 10 subfamilies) (D. R. Robinson et al., Oncogene, 2000, 19: 5548-5557, which is incorporated herein by reference in its entirety).

Transmembrane protein tyrosine kinases are receptors for many growth factors. Binding of a growth factor to a tyrosine kinase receptor activates the kinase, which triggers the transfer of a phosphate group from an ATP molecule to selected tyrosine residues of the receptor itself (auto-phosphorylation) as well as to selected tyrosine residues of specific substrate molecules that play a role in signaling pathways (for a more complete description of the mechanism, see, for example, J. Schlessinger and A. Ullrich, Neuron. 1992, 9: 303-391). Examples of growth factors associated with tyrosine kinase receptors include epidermal growth factors, platelet-derived growth factors, fibroblast growth factors, hepatocyte growth factors, insulin and insulin-like growth factors, nerve growth factors, vascular endothelial growth factors, and colony-stimulating factors. Compared to tyrosine kinase receptors, intracellular protein tyrosine kinases lack extracellular and transmembrane regions. They generally function by interacting and forming complexes with intracellular domains of cell-surface receptors. Cytokines and hormones are receptor ligands that signal through intracellular tyrosine kinases.

Tyrosine kinases whose phosphorylating activity can be assessed by the methods of the invention may be any member of the transmembrane tyrosine kinase family or any member of the intracellular tyrosine kinase family (for a list and classification of families and subfamilies of tyrosine kinases, see, for example, D. R. Robinson et al., Oncogene, 2000, 19: 5548-5557, which is incorporated herein by reference in its entirety).

Suitable tyrosine kinase receptors may be selected, for example, among members of the ALK (anaplastic lymphoma kinase), AXL or ARK (adhesion-related kinase), DDR (discoidin domain receptor), EGFR (epidermal growth factor receptor), EPH (ephrin receptor), FGFR (fibroblast growth factor receptor), INSR (insulin receptor kinase), MET, MUSK (muscle specific kinase), PDGFR (platelet-derived growth factor receptor), PTK7 (protein tyrosine kinase 7), RET, ROR (receptor tyrosine kinase-like orphan receptor), ROS, RYK (atypical orphan receptor tyrosine kinase), TIE, TRK (tropomyosin-related kinase), VEGFR (vascular endothelial growth factor receptor), and AATYK (apoptosis-associated tyrosine kinase) subfamilies.

For example, a tyrosine kinase receptor may be selected among members of the PDGFR subfamily, which includes PDGFRα, PDGFRβ, CSFIR, c-Kit and c-fms. These receptors consist of glycosylated extracellular domains composed of variable numbers of immunoglobulin-like loops and an intracellular region wherein the tyrosine kinase domain is interrupted by unrelated amino acid sequences. Other examples of suitable tyrosine kinase receptors whose phosphorylating activity can be studied by the assays of the invention include members of the VEGFR subfamily, which contains VEGFR1, VEGFR2 and VEGFR3. VEGFRs are dimeric glycoproteins which are similar to PDGFRs but have different biological functions. In particular, VEGFRs are presently thought to play a central role in vasculogenesis and angiogenesis.

Suitable intracellular (non-receptor) tyrosine kinases for use in the practice of the inventive methods may be selected among members of the ABL (Abelson tyrosine kinase), ACK (acetate kinase), CSK (C-terminal Src kinase), FAK (focal adhesion kinase), FES, FRK (fyn-related kinase), JAK (Janus kinase), SCR, TEC and SYK (spleen tyrosine kinase) subfamilies.

For example, an intracellular tyrosine kinase may be selected from the SRC subfamily, which is so far the largest group of non-receptor protein tyrosine kinases and which includes Src, Yes, Fyn, Lyn, Lck, Blk, Hck, Fgr and Yrk. Members of the SRC subfamily have been reported to be implicated in many signal transduction pathways, such as those involved in neuronal development and B-cell development. Other suitable intracellular tyrosine kinases include members of the JAK subfamily, which includes Jak1, Jak2, Tyk2 and Jak3. JAKs are known to play a critical role in cytokine signaling. Other examples of intracellular tyrosine proteins that can be studied by the methods of the invention are members of the SYK subfamily, including Syk and ZAP70, which are involved in cell activation.

Alternatively, screening methods of the invention may be developed that target a particular kinase of the serine/threonine kinase family. Enzymes of this class specifically phosphorylate serine or threonine residues of intracellular proteins and regulate a wide variety of cellular events, which include the ability of cells to enter and/or complete mitosis, cellular proliferation, cellular differentiation, the control of fat metabolism, immune responses, inflammatory responses, and the control of glycogen metabolism. The serine/threonine kinases are predominantly non-receptors although there are a few transmembrane serine/threonine protein kinases. Members of the serine/threonine kinase family are activated by diverse stimuli ranging from cytokines, growth factors, neurotransmitters, hormones, cellular stress to cell adherence.

Serine/threonine protein kinases whose phosphorylating activity can be assessed by the methods of the invention include members of the AGC (cyclic nucleotide dependent kinase), CMGC and CAMK (calcium/calmodulin-dependent protein kinase) families.

Members of the AGC family are functionally and structurally well conserved. The AGC family includes different subfamilies of serine/threonine kinases such as, for example, the AKT or PKB (protein kinase B) subfamily, PKA (cAMP-dependent kinase) subfamily, SGK (serum/glucocorticoid regulated kinase) subfamily, PKC (protein kinase C) subfamily, PDPK/PDK (phosphoinositide-dependent protein kinase) subfamily, DMPK (dystrophia myotonic-protein kinase) subfamily and S6K (ribosomal protein S6 kinase) subfamily. CMGC is an acronym based on the names of the best characterized subfamilies of this serine/threonine kinase family, namely CDK (cyclin-dependent protein kinase) subfamily, MAPK/ERK (mitogen-activated protein kinase/extracellular signal regulated kinase) subfamilies, GSK3 (glycogen-synthase kinase 3) subfamily, and CKII (casein kinase II) subfamily (“The Protein Kinase Facts Book: Protein-Serine Kinases”, G. Hardie and S. Hanks (Eds.), 1995, Academic Press, Inc.: San Diego, Calif.). The CAMK family of serine/threonine kinases includes, but is not limited to, the CaMK I/IV subfamily, CaMK II subfamily, MAGUK (or CASK, calcium/calmodulin-dependent serine protein kinase) subfamily, and DCaMKL (double cortin and calcium/calmodulin-dependent protein kinase) subfamily.

For example, a suitable serine/threonine kinase for use in the practice of the methods of the invention may be selected from the MAP kinase family. MAP kinases are activated by a variety of signals, including growth factors, cytokines, UV radiation, and stress-inducing agents. MAP kinases phosphorylate various substrates including transcription factors, which in turn regulate the expression of specific sets of genes and thus mediate a specific response to the stimulus. Other suitable serine/threonine kinases are members of the CDK family. CDKs consist of a β-sheet rich amino-terminal lobe and a larger carboxy-terminal lobe that is mostly α-helical. The CDKs display the 11 subdomains shared by most protein kinases and range in molecular mass from 33 to 44 kDaltons. This subfamily of kinases, which includes CDK1, CDK2, CDK4 and CDK6, requires phosphorylation at the residue corresponding to CDK2 Thr160 in order to be fully active (L. Meijer, Drug Resistance Updates, 2000, 3: 83-88). Each CDK complex is formed from a regulatory cyclin subunit (e.g., cyclin A, B1, B2, D1, D2, D3 and E) and a catalytic kinase subunit (e.g., CDK1, CDK2, CDK4, CbK5 and CDK6). Each different kinase/cyclin pair functions to regulate the different and specific phases of the cell cycle known as the G1, S, G2 and M phases (E. Nigg, Nature Reviews, 2001, 2: 21-32; P. Flatt and J. Pietenpol, Drug Metab. Rev. 2000, 32: 283-305).

Alternatively, screening methods of the invention may be developed that target a particular kinase of the histidine kinase family. Histidine kinases were previously thought to exist only in prokaryotes. However, eukaryotic members of this superfamily have now been described (C. Chang et al., Science, 1993, 263: 539-544; I. M. Ota and A. Varshavsky, Science, 1993, 262: 566-569; and T. Maeda et al., Nature, 1994, 369: 242-245). Members of this family bear little homology with mammalian serine/threonine kinases or tyrosine kinases, and have distinctive sequence motifs of their own (J. R. Davie et al., J. Biol. Chem. 1995, 270: 19861-19871). Mammalian histidine kinases include, but are not limited to, PDK1, PDK3 and PDK4 (pyruvate dehydrogenase kinase 1, 3 and 4, respectively), and BCKDK (branched chain α-ketoacid dehydrogenase kinase).

Mitochondrial protein kinases have also been described that show structural homology to the histidine kinases, but phosphorylate their substrates on serine residues (K. M. Popov et al., J. Biol. Chem. 1992, 267: 13127-13130; and K. M. Popov et al., J. Biol. Chem. 1993, 268: 22602-22606). Several other protein kinases have been reported that show a lack of homology with either of the kinase superfamilies (Y. Maru and O. N. Witte, Cell, 1991, 67: 459-468; J. F. Beeler et al., Mol. Cell. Biol. 1994, 14: 982-988; R. Dikstein et al., Cell, 1996, 84: 781-790; L. M. Futey et al., J. Biol. Chem. 1995, 270: 523-529; and L. Eichinger et al., EMBO J. 1996, 15: 5547-5556). The activity of such protein kinases can also be studied by the methods of the invention.

Kinase Activator

In the methods of the invention, kinase activity is typically assessed by measuring the amount of a phosphorylated substrate. In the case of non-constitutively active kinases, phosphorylation of a substrate molecule occurs in response to an extracellular or other type of stimulus, herein termed “kinase activator”. Accordingly, in certain embodiments, the inventive assays include exposing the cells to a kinase activator such that activation of the kinase takes place and results in phosphorylation of the substrate.

A kinase activator for use in the practice of the methods of the invention may be any of a variety of stimuli including environmental stress signals, chemical stress signals, biochemical stimuli, and any combinations of such stimuli.

An environmental stress signal may be, for example, an osmotic shock. Osmotic shock (also called cold osmotic shock) may be administered, for example, by incubating cells in a hyperosmolar solution of inert solute (e.g., sucrose) containing ethylenediaminetetraacetic acid (EDTA) followed by centrifugation and suspension of the cells in cold water containing Mg 2+. Alternatively, an environmental stress signal may be a heat shock, which can be administered, for example, by heating the cells at 45° C. for 30 minutes. An environmental stress signal may, alternatively, be ultraviolet radiation, which can be administered, for example, using a UV-C germicidal bulb (254 nm) as described by Q. Zhan et al. (Mol. Cell Biol. 1993, 13: 4242-4250).

Chemical stimuli that can be used as kinase activators in the methods of the invention include oxidative stress, which is known to induce cell death in a wide variety of cell types, apparently by modulating intracellular signaling pathways. An oxidative stress treatment may be administered, for example, by adding hydrogen peroxide (H₂O₂) or diamine to cells. Human carcinogens, such as inorganic arsenic (e.g., sodium arsenite) and environmental pollutants, such as heavy metals (e.g., mercury, cadmium, and the like) may, alternatively, be used as chemical kinase activators.

A biochemical stimulus may be any of a variety of extracellular factors that induce activation of protein kinases, such as, for example, growth factors, cytokines, growth hormones, and neurotransmitters.

Growth factors are proteins that bind to receptors on the cell surface, with the primary result of activating cellular proliferation and/or differentiation. Many growth factors are quite versatile, stimulating cellular division in numerous different cell types; while others are specific to a particular cell type. Growth factors suitable for use as kinase activators in the methods of the invention include, but are not limited to, epidermal growth factors (EGFs, which promote proliferation of mesenchymal, glial and epithelial cells); fibroblast growth factors (FGFs, which promote proliferation of many cells, inhibit some stem cells, and induce mesoderm to form in early embryo); colony-stimulating factors (such as granulocyte-CSF (G-CSF), macrophage-CSF (M-CSF); and granulocyte-macrophage-CSF (GM-CSF)); hepatocyte growth factors (HGFs); insulin and insulin-like growth factors (IGFs and ILGFs, which promote proliferation of many cell types); nerve growth factors (NGFs, which promote neurite outgrowth and neural cell survival); platelet-derived growth factors (PDGFs, which promote proliferation of connective tissue, glial and smooth muscle cells); and vascular endothelial growth factors (VEGFs, which are thought to play a critical role in vasculogenesis and angiogenesis).

Cytokines are a unique family of growth factors. Secreted primarily from leukocytes, cytokines stimulate both the humoral and cellular immune responses, as well as the activation of phagocytic cells. Cytokines suitable for use as kinase activators in the methods of the invention include, but are not limited to, interleukins (such as IL-1, which is one of the most important immune-response modifying interleukins; IL-2, which is the major interleukin responsible for clonal T-cell proliferation; IL-6, which is produced by macrophages, fibroblasts, endothelial cells and activated T-helper cells; and IL-8, which exerts chemoattractant activity to leukocytes and fibroblasts); interferons (such as IFN-α and IFN-β, which are known as type I interferons and are predominantly responsible for the antiviral activities of the interferons); and tumor necrosis factors (such as TNF-α, which is a major immune response-modifying cytokine produced primarily by activated macrophages; and TNF-β, which is characterized by its ability to kill a number of different cell types as well as to induce terminal differentiation in others).

Alternatively, growth hormones may be used as kinase activators in the practice of the methods of the invention. The growth hormone family comprises human placental lactogen (hPL), growth hormone (GH) and prolactin (Prl). All contain about 200 amino acids, 2 sulfide bonds and no glycosylation. Although each has special receptors and unique characteristics to their activity, they all possess growth-promoting and lactogenic activity.

Other examples of suitable kinase activators are neurotransmitters, including, for example, acetylcholine, glycine, glutamate, γ-amino butyric acid (GABA), dopamine, norepinephrine (also called noradrenaline) and histamine. These neurotransmitters are hydrophilic molecules that bind to cell-surface receptors, thereby inducing conformational changes that open ion channels and create ion fluxes in the cell.

As can be appreciated by one of ordinary skill in the art, selection of a kinase activator for the development of an assay according to the present invention will be governed by the nature of the kinase whose phosphorylating activity is to be assessed. For example in Example 1, JAK3 is activated using Interleukin-2 (IL-2), while in Example 2, JAK2 is activated using GM-CSF.

The type and amount of kinase activator(s) to be added to each well will depend on the number of cells present in each well. In the methods of the invention, stimulation of non-constitutively active kinases is carried out by incubating the cells at 37° C. in a humidified incubator in a culture medium comprising a kinase activator. Generally, the concentration of kinase activator in the medium is between about 0.1 and about 1000 ng/mL. In Example 1, HT-2 cells are activated by incubation at 37° C. for 15 minutes in the presence of 10 ng/mL of IL-2. In Example 2, TF-1 cells are activated by incubation at 37° C. for 15 minutes in the presence of 2.5 ng/mL of rhGM-CSF.

Constitutively Active Kinases

In other embodiments, the kinase is constitutively active, i.e., it exhibits the ability to catalyze the phosphorylation of a substrate molecule in the absence of stimulation. Therefore, in these embodiments that relate to constitutively active kinases, the methods of the invention do not involve kinase stimulation using a kinase activator.

Constitutively active kinases may be endogenously expressed in cells or may be expressed by transfection. Endogenous constitutively active kinases may be Tel Jak2 or mutated kinases (e.g., Erk2, cMet, Akt, etc) which when activated lead to cancer.

Phosphorylated Substrate

In the methods of the invention, kinase activity is generally assessed by measuring the amount of phosphorylated substrate.

Intracellular signaling pathways, or protein kinase cascades, propagate extracellular signals received at the plasma membrane to the interior of the cell through a series of phosphorylating events. On average, a protein kinase phosphorylates at least 20 different substrates in vivo. Accordingly, in the methods of the invention, a substrate may be any of a wide variety of molecules that are involved in one or more signaling pathways and whose phosphorylation by the kinase ultimately results in the modification of one or more cellular responses.

It is estimated that approximately one third of all proteins in mammalian cells are phosphorylated at some time or another (H. Steen et al., J. Biol. Chem. 2002, 277: 1031-1039) and that the majority of human proteins may be phosphorylated at more than 100,000 sites (H. Zhang et al., J. Biol. Chem. 2002, 277: 39379-39387). Although many substrates of protein kinases are already known, fewer than 2,000 phosphorylation sites have been identified so far and there is considerable interest in proteomics to design and develop improved methods and techniques to identify, characterize and monitor new sites of protein phosphorylation.

In the methods of the invention, a phosphorylated substrate preferably contains at least one phosphorylated amino acid residue, such as a phosphorylated tyrosine residue, a phosphorylated serine residue, a phosphorylated threonine residue or a phosphorylated histidine residue. Substrate molecules may be large signaling proteins such as downstream transmembrane or intracellular protein kinases. Alternatively, substrate molecules may be intracellular target proteins such as metabolic enzymes (whose phosphorylation ultimately leads to altered cell metabolism), gene regulatory proteins (whose phosphorylation ultimately leads to altered gene expression) or cytoskeletal proteins (whose phosphorylation ultimately leads to altered cell shape or movement).

As will be readily recognized by one of ordinary skill in the art, a wide variety of kinase/substrate combinations may be investigated using the methods of the invention. Illustrative examples of such combinations are described below.

For example, members of the JNK family are known to be activated by proinflammatory cytokines, such as tumor necrosis factor-α (TNF-α) and interleukin-1β (IL-1β), as well as by environmental stress, including UV radiation, hypoxia, and osmotic shock (S. A. Minden et al., Biochem. Biophys. Acta, 1997, 1333: F85-F104). The downstream substrates of JNKs include transcription factors c-Jun, ATF-2, Elk1, p53 and a cell death domain protein (DENN) (H. Zhang et al., Proc. Natl. Acad. Sci. USA, 1998, 95: 2586-2591). Each JNK isoform binds to these substrates with different affinities, suggesting a regulation of signaling pathways by substrate specificity of different JNKs in vivo (S. Gupta et al., EMBO J. 1996, 15: 2760-2770).

While many cellular pathways propagate a signal from a cell-surface receptor to the nucleus through a long cascade of signaling/phosphorylating events, the JAK/STAT signaling pathway provides one of the most direct routes. Upon activation, the Janus kinases phosphorylate and activate a set of latent gene regulatory proteins called STATs (Signal Transducers and Activators of Transcription), which move into the nucleus and stimulate the transcription of specific genes.

FLT-3 and c-Kit, which belong to the family of type III receptor tyrosine kinases, play an important role in the maintenance of stem cell/early progenitor pools as well as in the development of mature lymphoid and myeloid cells (S. Lyman and S. Jacobsen, Blood, 1998, 91: 1101-1134). Both receptors contain an intrinsic kinase domain that is activated upon ligand-mediated dimerization of the receptors. Some of the proposed downstream regulators of FLT-3 and c-Kit receptor signaling include, PLCγ, PI3-kinase, Grb-2, SHIP and Src related kinases (B. Scheijen and J. D. Griffin, Oncogene, 2002, 21: 3314-3333).

Glycogen synthase kinase-3 (GSK-3), which is a serine/threonine kinase, has been implicated in various diseases including diabetes, Alzheimer's disease, CNS disorders and cardiomyocyte hypertrophy. These diseases are associated with the abnormal operation of certain cell signaling pathways in which GSK-3 plays a role. GSK-3 has been found to phosphorylate and modulate the activity of a number of regulatory proteins. These proteins include glycogen synthase, which is the rate limiting enzyme necessary for glycogen synthesis, the microtubule associated protein Tau, the gene transcription factor β-catenin, the translation initiation factor e1F2B as well as ATP citrate lyase, axin, heat shock factor-1, c-Jun, c-myc, c-myb, CREB, and CEPBα.

The Aurora family of serine/threonine kinases is essential for cell proliferation (J. R. Bischoff and G. D. Plowman, Trends Cell Biol 1999, 9: 454459; R. Giet and C. Prigent, J. Cell Sci. 1999, 112: 3591-3601; E. A. Nigg, Nat. Rev. Mol. Cell Biol. 2001, 2: 21-32; R. Adams et al., Trends Cell. Biol. 2001, 11: 49-54). In mammalian cells, proposed substrates for Aurora kinases include histone H3, a protein involved in chromosome condensation, and CENP-A, a myosin II regulatory light chain, protein phosphate 1, TPX2, all of which are required for cell division.

CaM kinase I was found to phosphorylate a variety of substrates including the neurotransmitter related proteins synapsin I and II, the gene transcription regulator, CREB, and the cystic fibrosis conductance regulator protein, CFTR (B. Haribabu et al., EMBO J. 1995, 14: 3679-3686), while CaM kinase IV is known to phosphorylate and activate the cyclic AMP response element binding proteins CREB and CREMτ (R. P. Matthews et al., Mol. Cell. Biol. 1994, 14: 6107-6116; P. Sun et al., Genes Dev. 1994, 8: 2527-2539; and H. Enslen et al., J. Biol. Chem. 1994, 269: 15220-15227).

Other examples of kinase/substrate combinations that can be studied by the methods of the invention include, but are not limited to, JAK3/STAT5, JAK2/STAT5, JNK1/GST-c-jun, JNK2/GST-c-jun, ERK1/myelin basic protein, ERK2/myelin basic protein, PKA/Kemptide, MEK-1/ERK-2, JNK2α2/ATF-2, JNK2α2/c-jun, SAPK-3/myelin basic protein, SAPK-4/myelin basic protein, and raf-1/MEK-1.

Detection of Phosphorylated Substrate—Fluorescently-Detectable Selective Probe

In the methods of the invention, the amount of phosphorylated substrate is determined using a fluorescently-detectable selective probe.

A selective probe may be any molecule, compound, factor, agent or moiety that exhibits a specific affinity for the phosphorylated substrate molecule of interest. The affinity for a phosphorylated substrate may be governed by physical forces such as ionic interactions, covalent bonding, as well as hydrophobic interactions or electrical potential. Preferred selective probes recognize and bind to certain types of phosphorylated substrates, for example to tyrosine-phosphorylated substrates.

A wide variety of selective probes may be used, including, but not limited to, biomolecules such as proteins, phospholipids, and DNA hybridizing probes. Due to their high degree of specificity for binding to a single molecular target in a mixture of molecules as complex as a cell, preferred selective probes are phospho-specific antibodies.

Phospho-Specific Antibody

In certain embodiments, exposing the cells to a fluorescently-detectable selective probe comprises adding to the cells a phospho-specific antibody that is directly or indirectly detectable by fluorescence. In these embodiments, the phospho-specific antibody specifically recognizes and binds to one or more phosphorylated residues of the phosphorylated substrate molecule. Preferably, the phosphorylated residue that is recognized by the specific antibody is a phosphorylated tyrosine, a phosphorylated serine, a phosphorylated threonine or a phosphorylated histidine.

Suitable antibodies may be any intact immunoglobulin molecules or fragments thereof (i.e., active portions of immunoglobulin molecules) that are capable of specifically recognizing and binding to an epitope of a phosphorylated substrate molecule. The type of antibody that can be used in the inventive kinase assays may be either monoclonal (recognizing one epitope of its target) or polyclonal (recognizing multiple epitopes). Preferably, antibodies are monoclonal.

Phospho-specific antibodies for use in the practice of the assay and screening methods of the invention may be produced or purchased from different commercial resources (see below). As will be appreciated by one of ordinary skill in the art, any type of antibody can be generated and/or modified to specifically recognize and bind to an epitope of a substrate molecule phosphorylated at one or more tyrosine, serine, threonine or histidine residues.

Methods for producing custom polyclonal antibodies are well known in the art and include standard procedures such as immunization of rabbits or mice with pure protein or peptide (see, for example, R. G. Mage and E. Lamoyi, in “Monoclonal Antibody Production Techniques and Applications”, 1987, Marcel Dekker, Inc.: New York, pp. 79-97). Anti-phosphotyrosine polyclonal antibodies can, for example, be made using the techniques described by M. F. White and J. M. Backer (as described in Methods in Enzymology, 1991, 201: 65-67, which is incorporated herein by reference in its entirety).

Monoclonal antibodies that specifically bind to a phosphorylated substrate may be prepared using any technique that provides for the production of antibody molecules by continuous cell lines in culture. These techniques include, but are not limited to, the hydroma technique, the human B-cell hydroma technique, and the EBV-hydroma technique (see, for example, G. Kohler and C. Milstein, Nature, 1975, 256: 495-497; D. Kozbor et al., J. Immunol. Methods, 1985, 81: 31-42; and R. J. Cote et al., Proc. Natl. Acad. Sci. 1983, 80: 2026-2030). Monoclonal antibodies may also be made by recombinant DNA methods (see, for example, U.S. Pat. No. 4,816,567). Other methods have been reported and can be employed to produce monoclonal antibodies for use in the practice of the invention (see, for example, R. A. Lerner, Nature, 1982, 299: 593-596; A. C. Nairn et al., Nature, 1982, 299: 734-736; A. J. Czemik et al., Methods Enzymol. 1991, 201: 264-283; A. J. Czernik et al., Neuromethods: Regulatory Protein Modification: Techniques & Protocols, 1997, 30: 219-250; A. J. Czernik et al., Neuroprotocols, 1995, 6: 56-61; and H. Zhang et al., J. Biol. Chem. 2002, 277: 39379-39387).

Techniques developed for the production of chimeric antibodies, the slicing of mouse antibody genes to human antibody genes to obtain a molecule with appropriate specificity and biological activity, can, alternatively, be used in the preparation of antibodies (S. L. Morrison et al., Proc. Natl. Acad. Sci., 1984, 81: 6851-6855; M. S. Neuberger et al., Nature, 1984, 312: 604-608; S. Takeda et al., Nature, 1985, 314: 452-454). Monoclonal and other antibodies can also be “humanized”; sequence differences between rodent antibodies and human sequences can be minimized by replacing residues which differ from those in the human sequences by site-directed mutagenesis of individual residues or by grafting of entire complementarity determining regions. Humanized antibodies can also be produced using recombinant methods (see, for example, GB 2 188 638 B).

Antibodies to be used in the methods of the invention can be purified by methods well known in the art (see, for example, S. A. Minden, “Monoclonal Antibody Purification”, 1996, IBC Biomedical Library Series: Southbridge, Mass.). For example, antibodies can be affinity-purified by passage over a column to which a phosphorylated substrate molecule is bound. The bound antibodies can then be eluted from the column using a buffer with a high salt concentration.

Instead of being prepared, phospho-specific antibodies may be purchased, for example, from BD Biosciences/Pharmingen (San Diego, Calif.); Upstate Biologicals, Inc. (Lake Placid, N.Y.), Bethyl Laboratories, Inc. (Montgomery, Tex.), Alexis Biochemicals (San Diego, Calif.), Sigma-Genosys (The Woodlands, Tex.), Affinity BioReagents, Inc. (Golden, Colo.), Cell Signaling (Beverly, Mass.), New England Biolabs, Inc. (Beverly, Mass.), Covance Research Products, Inc. (Berkeley, Calif.), and Stressgen Biotechnologies Corp. (Victoria, BC, Canada).

The amount of phospho-specific antibody to be added per well will depend primarily on its avidity for the phosphorylated substrate molecule and on the number of cells present per well. Such amount can easily be determined by one of ordinary skill in the art.

Fluorescent Label

In certain embodiments, the amount of phosphorylated substrate is determined using a phospho-specific antibody linked to a fluorescent label. In other embodiments, the amount of phosphorylated substrate is determined using a phospho-specific antibody and a secondary antibody linked to a fluorescent label. The role of the fluorescent label is to allow detection and visualization of the binding of the specific antibody to the phosphorylated substrate. Preferably, the fluorescent label is selected such that it generates a signal which can be measured and whose intensity is related (e.g., proportional) to the amount of specific antibody bound to the phosphorylated substrate.

Favorable optical properties of fluorescent labeling agents to be used in the practice of the invention include high molar absorption coefficient, high fluorescence quantum yield, and photostability. Preferred fluorescent dyes exhibit absorption and emission wavelengths in the visible (i.e., between 400 and 700 nm) or the near infra-red (i.e., between 700 and 950 nm) rather than in the ultraviolet range (i.e., below 400 nm) of the spectrum to avoid possible interference from the candidate compound(s) to be screened. Selection of a particular fluorescent label will be governed by the nature and characteristics of the illumination and detection systems within the Flow Cytometry Plate Reader used in the assay. More specifically, a suitable fluorescent label is one that can be efficiently excited by the light beam of the plate reader device and whose emission can be efficiently detected by its detector.

Numerous fluorescent labels of a wide variety of structures and characteristics are suitable for use in the practice of the present invention. Suitable fluorescent labels include, but are not limited to, quantum dots (i.e., fluorescent inorganic semiconductor nanocrystals) and fluorescent dyes such as Texas red, fluorescein isothiocyanate (FITC), phycoerythrin (PE), rhodamine, fluorescein, carbocyanine, Cy-3™ and Cy-5™ (i.e., 3- and 5-N,N′-diethyltetra-methylindodicarbocyanine, respectively), merocyanine, styryl dye, oxonol dye, BODIPY dye (i.e., boron dipyrromethene difluoride fluorophore), and analogues, derivatives or combinations of these molecules.

The association between the phospho-specific antibody (or between the secondary antibody) and fluorescent label can be covalent or non-covalent. Preferably, the association is covalent. More preferably, in order to permit quantitative studies, a defined number of fluorescent label molecules are covalently attached to a single molecule of antibody (e.g., one fluorescent label per antibody). Fluorescently-labeled antibodies can be prepared by incorporation of or conjugation to a fluorescent dye. Fluorescent labels can be attached to the antibody either directly or indirectly through a linker. Linkers or spacer arms of various lengths are known in the art and are commercially available. Such linkers can, for example, be selected to reduce steric hindrance. Preferably, attachment of a fluorescent label to a phospho-specific antibody or to a secondary antibody does not significantly affect the specific binding activity of the antibody.

Methods for fluorescently-labeling antibodies are well-known in the art. Fluorescent dyes are usually commercially available as NHS-esters, maleimides, and hydrazides to make them suitable for labeling via reaction with different chemical groups such as amine, thiol and aldehyde groups, respectively. Fluorescent labeling dyes as well as labeling kits are commercially available from, for example, Amersham Biosciences Inc. (Piscataway, N.J.), Molecular Probes Inc. (Eugene, Oreg.), Prozyme, Inc. (San Leandro, Calif.) and New England Biolabs Inc. (Berverly, Mass.).

Alternatively, fluorescently-labeled phospho-specific antibodies may be purchased from, for example, from BD Biosciences/Pharmingen (San Diego, Calif.) and AnaSpec (San Jose, Calif.). Fluorescently-labeled secondary antibodies are also commercially available, for example, from Santa Cruz Biotechnology (Santa Cruz, Calif.), Jackson ImmunoResearch Labs Inc. (West Grove, Pa.), and Rockland Immunochemicals Inc. (Gilbertsville, Pa.).

Selection of a particular fluorescent label and/or labeling technique will depend on the situation and will be governed by several factors, such as the ease and cost of the labeling method, the quality of sample labeling desired, the effects of the fluorescent label on the binding of the antibody (e.g., on the rate and/or efficiency of the binding process), the nature of the illumination and detection systems of the Flow Cytometry Plate Reader to be used, the nature and intensity of the signal generated by the fluorescent label, and the like.

Flow Cytometry Plate Reader

The assay and screening methods of the invention include measuring the amount of fluorescently-detectable selective probe bound to a phosphorylated substrate molecule preferably using a Flow Cytometry Plate Reader.

Conventional analysis platforms for cell-based assays fall into two general groups: macro-imagers which view a large number of samples in a whole assay microplate (thus providing a “well-by-well analysis”) and micro-imagers which have sufficient resolution to image individual cells in a sample (thus providing a “cell-by-cell analysis”). The former systems, which allow for rapid analysis of large numbers of cell samples, have found a wide variety of applications in the biotechnology and pharmaceutical industry, especially in high-throughput drug screening. However, data obtained using these systems correspond to measurements of the average response or average characteristic of a population of cells rather than reflect behaviors or properties of individual cells. Micro-imagers, on the other hand, provide multi-parameter data at the cellular or sub-cellular levels, lead to detailed information about the temporal-spatial dynamics of cell constituents and processes, and allow differences in characteristics or in responses between cells to be analyzed. These systems generally extract multicolor fluorescence information derived from specific fluorescence-based reagents incorporated into cells (K. A. Giuliano et al., in “In Optical Microscopy for Biology”, B. Herman and K. Jacobson (Eds.), 1990, Wiley-Liss: New York, pp. 543-557; K. Hahn et al., Nature, 1992, 359: 736-738; D. L. Farkas et al., Ann. Rev. Physiol. 1993, 55: 785-817; K. A. Giuliano et al., Ann. Rev. Biophys. Biomol. Struct. 1995, 24: 405-434; and A. Waggoner et al., Hum. Pathol. 1996, 27: 494-502). However, due to technical limitations, these micro-imager systems have not yet been widely applied to high-throughput screening.

The Flow Cytometry Plate Reader to be used in the practice of the methods of the invention combines the advantages of both types of analysis platforms as it can perform a multi-parametric cell-by-cell flow cytometric analysis of a large number of cell samples in a short period of time.

Flow cytometry is a sensitive and quantitative technique that analyzes particles (such as cells) in a fluid medium based on the particles' optical characteristics (for background information on flow cytometry, see, for example, H. M. Shapiro, “Practical Flow Cytometry”, 3^{rd} Ed., 1995, Alan R. Liss, Inc.; and “Flow Cytometry and Sorting, Second Edition”, Melamed et al. (Eds), 1990, Wiley-Liss: New York, which are incorporated herein by reference in their entirety). The fundamental concept of flow cytometry is simple. A flow cytometer hydrodynamically focuses a fluid suspension of particles which have been attached to one or more flurorophores, into a thin stream so that the particles flow down the stream in substantially single file and pass through an examination or analysis zone. A focused light beam, such as a laser beam, illuminates the particles as they flow through the examination zone. Optical detectors within the flow cytometer measure certain characteristics of the light as it interacts with the particles. Light interaction with the particles is generally measured as light scatter and particle fluorescence at one or more wavelengths.

Since the 1960's, standard flow cytometry has been widely used for studying a variety of phenotypic, biochemical and molecular characteristics of cells at the single cell level (J. P. Nolan and L. A. Sklar, Nature Biotech. 1998, 16: 633-638). Cells to be analyzed by flow cytometry are usually stained with one or more fluorescent labels specific for cell components of interest. Light scatter measurements provide information regarding properties such as cell size, cell shape, and cytoplasmic granularity. Fluorescence measurements allow one to determine, with high accuracy, relative quantities of a variety of cell constituents simultaneously. Furthermore, when the measurements are recorded in a list mode, it is possible to attribute each of these features on a cell-by-cell basis. Cellular heterogeneity can thus be estimated and subpopulations with distinct characteristics can be identified. Thus, multi-parameter flow cytometry offers opportunities to describe the complex relationships between different cellular processes.

Applications of standard flow cytometry have included determination of protein, lipid, DNA, and RNA product content, determination of target cells against particulate background, evaluation of antibiotic effects, determination of viability, and assessment of DNA degradation (apoptosis). Flow cytometry has also been used in fields as diverse as ligand binding and enzyme kinetics, cell cycle analysis, diagnostics and detection of soluble agents, phenotypic analysis of intracellular or extracellular markers, and analysis of GFP expression in mammalian cells.

In particular, standard flow cytometry has been shown to provide a rapid and efficient way to measure kinase activity and study kinase cascades in individual cells (see, for example, P. O. Krutzik and G. P. Nolan, Cytometry, 2003, 55A: 61-70; D. H. Hickerson and A. P. Bode, Hematol. Oncol. Clin. North Am. 2002, 16: 421-454; O. D. Perez and G. P. Nolan, Nature Biotechnology, 2002, 20: 155-162; S. Chow et al., Cytometry, 2001, 46: 72-78; G. Uzel et al., Clin. Immunol. 2001, 100: 270-276; F. Lund-Johansen et al., Cytometry, 2000, 39: 250-259; V. C. Maino and L. J Picker, Cytometry, 1998, 34: 207-215; C. Prussin, J. Clin. Immunol. 1997, 17: 195-204; and P. Hubert et al., Cytometry, 1997, 29: 83-91, which are incorporated herein by reference in their entirety).

The Flow Cytometry Plate Reader used in the assay and screening methods of the invention allows the same cell-by-cell, multi-parameter measurements to be performed than traditional flow cytometry instruments. However, unlike traditional flow cytometry instruments, the Flow Cytometry Plate Reader can carry out such cell-by-cell analysis for a large number of cell samples in a short period of time. Applied to drug screening according to the methods of the invention, such a Plate Reader allows a more efficient validation of cellular targets, a higher capacity for predictive toxicology and a more effective lead optimization, which decreases cycle times for drug discovery while increasing the probability of success in pre-clinical and clinical trials.

As described in Examples 1 and 2, a preferred Flow Cytometry Plate Reader system used by the Applicants is the Guava Personal Cell Analyzer (PCA)-96 that was developed by Guava Technologies (Hayward, Calif.). This system, which is based on patented micro-capillary technology (see U.S. Pat. No. 6,710,871 and U.S. Pat. Appl. Nos. 2002/0028434 and 2004/0036870), requires only a few microliters of sample volume, thus reducing cost by saving precious or expensive cells, reagents and candidate compounds and minimizing generation of bio-hazardous waste. Furthermore, the instrument provides results rapidly with a process time of 30 to 50 minutes by 96-well plate.

Various parameters of the cells can be measured with the Guava PCA-96 using a forward scatter and two fluorescent detection channels. Data generated by the Guava PCA-96 software may be saved in FCS (Flow Cytometry Standard) 2.0 or 3.0 format. Files in FCS format can be read by third party flow cytometry analysis software such as FCS Express, Win MDI, ModFit, and the like. In addition, data summaries are also stored in CSV database format readable by spreadsheet software such as Microsoft Excel. Furthermore, the Guava instrument may be integrated with laboratory automation equipment products such as the Hudson Control PlateCrane (commercialized by Hudson Control Group, Inc., Springfield, N.J.).

II. Screening of Candidate Compounds and Identification of Modulators of Kinase Activity

In another aspect, the invention relates to screening methods for identifying modulators of kinase activity. In particular, assays are described that allow compounds or agents to be tested for their ability to inhibit or enhance the phosphorylating activity of a given kinase inside a cell.

More specifically, a method is provided for identifying compounds that have the ability to modulate the phosphorylating activity of an enzyme in cells, wherein the enzyme is a protein kinase catalyzing the phosphorylation of a substrate molecule that is involved in a signaling pathway. The inventive method comprises steps of: providing cells in a plurality of wells of a multi-well assay plate; incubating cells in some wells of the assay plate with a candidate compound under conditions and for a time sufficient to allow equilibration, thus obtaining test cells; incubating cells in other wells of the assay plate under the same conditions and for the same time in the absence of the candidate compound, thus obtaining control cells; exposing the test and control cells to a fluorescently-detectable selective probe such that the selective probe binds to the phosphorylated substrate; measuring the amount of selective probe bound to the phosphorylated substrate in the test and control cells using a Flow Cytometry Plate Reader; comparing the amount of bound probe in the test and control cells; and determining that the candidate compound modulates the phosphorylating activity of the kinase if the amount of bound probe in the test cells is less than or greater than the amount of bound probe in the control cells.

The cell systems, kinases, kinase activators, phospho-specific antibodies, fluorescent labels and experimental conditions described above are also suitable for use in the practice of the screening methods of the invention.

Candidate Compounds or Agents

The screening methods of the invention may be used for identifying compounds or agents that have the ability to modulate or alter the phosphorylating activity of a kinase of interest inside a cell. Screening according to the present invention is generally performed with the goal of developing modulators of kinase activity for therapeutic purposes. In certain embodiments, the inventive methods are used for identifying compounds or agents that inhibit or suppress the phosphorylating activity of a kinase of interest. In other embodiments, the inventive methods are used for identifying compounds or agents that enhance or stimulate the phosphorylating activity of a kinase of interest.

As will be appreciated by those of ordinary skill in the art, any kind of compounds or agents can be tested and screened using the inventive methods. A candidate compound may be a synthetic or natural compound; it may be a single molecule or a mixture of different molecules. In certain embodiments, the inventive methods are used for testing one or more compounds. In other embodiments, the inventive methods are used for screening collections or libraries of compounds. As used herein, the term “collection” refers to any set of compounds, molecules or agents, while the term “library” refers to any set of compounds, molecules or agents that are structural analogs.

Traditional approaches to the identification and characterization of new and useful drug candidates generally include the generation of large collections and/or libraries of compounds followed by testing against known or unknown targets (see, for example, WO 94/24314; WO 95/12608; M. A. Gallop et al., J. Med. Chem. 1994, 37: 1233-1251; and E. M. Gordon et al., J. Med. Chem. 1994, 37: 1385-1401). Both natural products and chemical compounds may be tested by the methods of the invention.

Natural product collections are generally derived from microorganisms, animals, plants, or marine organisms; they include polyketides, non-ribosomal peptides, and/or variants (non-naturally occurring) thereof (for a review, see, for example, D. E. Cane et al., Science, 1998, 82: 63-68). Chemical libraries often consist of structural analogs of known compounds or compounds that are identified as “hits” or “leads” via natural product screening. Chemical libraries are relatively easy to prepare by traditional automated synthesis, PCR, cloning or proprietary synthetic methods (see, for example, S. H. DeWitt et al., Proc. Natl. Acad. Sci. U.S.A. 1993, 90:6909-6913; E. Erb et al., Proc. Natl. Acad. Sci. U.S.A. 1994; 91: 11422-11426; R. N. Zuckermann et al., J. Med. Chem. 1994, 37: 2678-2685; C. Y. Cho et al., Science, 1993, 261: 1303-1305; Carell et al., Angew. Chem. Int. Ed. Engl. 1994, 33: 2059-2060; Carell et al., Angew. Chem. Int. Ed. Engl. 1994, 33: 2061-2063; and M. A. Gallop et al., J. Med. Chem. 1994, 37: 1233-1251; and P. L. Myers, Curr. Opin. Biotechnol. 1997, 8: 701-707).

Collections of natural compounds in the form of bacterial, fungal, plant and animal extracts are available from, for example, Pan Laboratories (Bothell, Wash.) or MycoSearch (Durham, N.C.). Libraries of candidate compounds that can be used in the practice of the present invention may be either prepared or purchased from a number of companies. Synthetic compound libraries are commercially available from, for example, Comgenex (Princeton, N.J.), Brandon Associates (Merrimack, N.H.), Microsource (New Milford, Conn.), and Aldrich (Milwaukee, Wis.). Libraries of candidate compounds have also been developed by and are commercially available from large chemical companies, including, for example, Merck, Glaxo Welcome, Bristol-Meyers-Squibb, Novartis, Monsanto/Searle, and Pharmacia UpJohn. Additionally, natural collections, synthetically produced libraries and compounds are readily modified through conventional chemical, physical, and biochemical means.

Useful modulators of kinase activity may be found within numerous classes of chemicals, including heterocycles, peptides, saccharides, steroids, and the like. In certain embodiments, the methods of the invention are used for identifying compounds or agents that are small molecules. In other embodiments, the inventive methods are used for screening small molecule libraries. Preferred small organic molecules have a molecular weight of more than 50 and less than about 2,500 Daltons; preferably less than 600-700 Daltons; more preferably less than 350 Daltons.

Candidate compounds to be tested and screened by the assays of the invention can be compounds previously unknown to have any pharmacological activity, or can be pharmacologic agents already known in the art. In particular, candidate compounds can be selected among agents or derivatives of agents already known in the art to modulate kinase activity. For example, the purine ring system is considered as a good starting point in the search for inhibitors of various protein kinases and a 2,6,9-trisubstituted purine library has been developed for such purposes (see, for example, P. Shultz, Science, 1998, 281: 533-538; and Y. T. Chang et al., Chem Biol. 1999, 6: 361-375). Similarly, the conserved and extremely well characterized nature of the ATP binding pocket has made it the most common, and most successful, target for kinase inhibition. Thus, libraries of compounds targeting ATP have been generated and can be used in the screening methods of the invention. Alternatively, candidate compounds can be selected among drugs or derivatives of drugs known in the art to be useful in the treatment of diseases or pathophysiological conditions associated or suspected to be associated with abnormal cellular responses triggered by kinase-mediated events.

The screening of small molecule libraries according to the assays of the invention provides “hits” or “leads”, i.e., compounds that possess a desired but not optimized biological activity. Test compounds identified by the methods of the invention as modulators of kinase activity may be modified to enhance efficacy, stability, pharmaceutical compatibility, and the like, in order to provide improved drug candidates. For example, test compounds identified by the inventive screening methods may be subjected to a structure-activity relationship (SAR) analysis. In such analyses, molecular structure and biological activity are correlated by observing the results of systemic structural modifications on defined biological endpoints. For example, comparison of the modulating effects of structurally-related compounds may help identify positions on candidate molecules that are important for their ability to inhibit or enhance the phosphorylating activity of a kinase of interest. Similarly, analysis of the effects of the stereochemistry of these compounds (i.e., the arrangement of their atoms in space) on their ability to modulate the phosphorylating activity of a given kinase may help identify conformations that are favorable to the inhibition or enhancement of kinase activity. Structure-activity relationship information available from the first round(s) of screening can then be used to generate small secondary libraries which are subsequently screened for compounds with higher activity.

Identification of Modulators of Kinase Activity

According to the screening methods of the invention, determination of the ability of a candidate compound to alter or modulate the phosphorylating activity of a given kinase inside a cell includes comparison of the amount of phosphorylated substrate in test cells and control cells. Test cells are incubated in the presence of the candidate compound to be studied, while control cells are incubated under the same conditions and for the same period of time except for the presence of the candidate compound. Both test and control cells then undergo the same treatments (including cell starvation and kinase activation in the case of non-constitutively active protein kinases, fixation, permeabilization, and staining) before analysis.

A candidate compound is identified as an inhibitor of the phosphorylating activity of a kinase if the amount of phosphorylated substrate in the test cells is less than the amount of phosphorylated substrate in the control cells. A candidate compound is identified as a stimulator of the phosphorylating activity of a kinase if the amount of phosphorylated substrate in the test cells is greater than the amount of phosphorylated substrate in the control cells.

Reproducibility of the results may be tested by incubating cells in more than one well of the assay plate (for example, in triplicate) with the same concentration of the same candidate compound. Additionally, since candidate compounds may be effective at varying concentrations depending on the nature of the compound and the nature of its mechanism(s) of action, varying concentrations of the candidate compound may be added to different wells containing cells. Generally, concentrations from about 1 fM to about 10 mM are used for screening. Preferred screening concentrations are between about 10 pM and about 100 μM. Furthermore, screening different concentrations of a candidate compound according to the methods of the invention allows the IC₅₀ value to be determined for that compound.

In certain embodiments, the methods of the invention further involve the use of one or more negative or positive control compounds. A positive control compound may be any molecule, agent, moiety or drug that is known to modulate the phosphorylating activity of the kinase under investigation in the screening method. A negative control compound may be any molecule, agent, moiety or drug that is known to have no significant effects on the phosphorylating activity of the kinase under investigation in the screening method. In these embodiments, the inventive methods further comprise comparing the modulating effects of the candidate compound to the modulating effects (or absence thereof) of the positive or negative control compound. Such negative and positive control compounds are known in the art (see, for example, S. P. Davies et al., Biochem. J. 2002, 351: 95-105; and J. Bain et al., Biochem. J. 2003, 371: 199-204) or may be identified by the methods described herein or by other kinase assays.

Using the methods of the invention, a candidate compound may be tested for its ability to modulate the phosphorylating activity of a tyrosine kinase, a serine/threonine kinase, a histidine kinase, or a dual-specificity kinase. A compound identified as a modulator of the phosphorylating activity of a kinase of interest may inhibit or enhance the kinase activity through a single mechanism of action. Alternatively, it may inhibit or enhance the kinase activity through a combination of different mechanisms of action. For example, the test compound may inhibit (e.g., by precluding, reversing or disrupting) the binding of the kinase activator to its cell-surface receptor. Alternatively, the test compound may favor or stimulate the binding of the kinase activator to its cell-surface receptor. The test compound may, additionally or alternatively, prevent or favor activation of a downstream intracellular protein kinase and/or it may affect the transfer of a phosphate group to a substrate molecule.

III. Pharmaceutical and Clinical Applications of Modulators of Kinase Activity

In another aspect, the present invention is directed to modulators of kinase activity. More specifically, the invention provides compounds identified by the screening methods as inhibitors or stimulators of the phosphorylating activity of a given protein kinase in cells.

Modulators of Kinase Activity as Therapeutic Agents

As mentioned above, various medical conditions are associated with abnormal cellular responses triggered by kinase-mediated events. Agents that have the ability to alter or affect such kinase-mediated events thereby inhibiting or suppressing the corresponding abnormal cellular responses may be beneficial in the prevention or treatment of diseases or pathophysiological conditions associated with these abnormal cellular responses. Such diseases and pathophysiological conditions include, but are not limited to, autoimmune diseases, inflammatory diseases, bone diseases, metabolic diseases, neurological and neurodegenerative diseases, cancer, cardiovascular diseases, allergies and asthma, and hormone-related diseases.

The screening methods of the invention may be used to identify, test and/or develop drugs with various clinical applications. Accordingly, the present invention provides compounds identified by the inventive screening methods as modulators of kinase activity. More specifically, compounds are provided that have the ability to inhibit or enhance the phosphorylating activity of a tyrosine kinase inside a cell. Other compounds provided by the present invention have the ability to inhibit or enhance the phosphorylating activity of a serine/threonine kinase inside a cell. Still other compounds provided herein have the ability to inhibit or enhance the phosphorylating activity of a histidine kinase inside a cell. Yet other compounds are provided that have the ability to inhibit or enhance the phosphorylating ability of more than one type of protein kinases.

For example, using inventive assays that target kinases of the JAK (Janus kinase) family, potential drugs with a variety of different therapeutic applications may be identified and developed. JAKs, which include JAK1, JAK2, JAK3 and TYK2, are tyrosine kinases that play a critical role in cytokine signaling. The downstream substrates of the JAK family of kinases include the Signal Transducer and Activator of Transcription (STAT) proteins. JAK/STAT signaling has been implicated in the mediation of many abnormal immune responses such as allergies (R. Malaviya et al., Biochem. Biophys. Res. Commun. 1999, 257: 807-813; R. Malaviya et al., J. Biol. Chem. 1999, 274: 27028-27038), asthma, autoimmune diseases, transplant rejection (R. A. Kirken, Transpl. Proc. 2001, 33: 3268-3270), rheumatoid arthritis (U. Muller-Ladner et al., J. Immunol. 2000, 164: 3894-3901), amyotrophic lateral sclerosis (V. N. Trieu et al., Biochem. Biophys. Res. Commun. 2000, 267: 22-25) and multiple sclerosis as well as in solid and hematologic malignancies such as leukemias (E. A. Sudbeck et al., Clin. Cancer Res. 1999, 5: 1569-1582) and lymphomas (P. R. Nielsen et al., Proc. Nat. Acad. Sci. U.S.A. 1997, 94: 6764-6769; C. L. Yu et al., J. Immunol. 1997, 159: 5206-5210; R. Catlett-Falcone et al., Immunity 1999, 10: 105-115). The pharmaceutical intervention in the JAK/STAT pathway has been reviewed (see, for example, D. A. Frank, Mol. Med. 1999, 5: 432456; and H. M. Seidel et al., Oncogene, 2000, 19: 2645-2656). Candidate compounds identified by the screening methods of the invention as modulators of the phosphorylating activity of kinases of the JAK family may be potentially useful therapeutic agents in the treatment of such diseases and clinical conditions.

Another important family of tyrosine kinases for which modulators may be identified by the inventive screening methods is the SRC family. Eight mammalian SRC family protein tyrosine kinases have been characterized to date: Src, Fyn, Yes, Fgr, Lyn, Hck, Lck and Blk. While Hck, Fgr, Blk and Lck are restricted to hematopoietic cell lineages, Lyn is expressed in these and neuronal tissues, and Src, Yes and Fyn are expressed ubiquitously (M. T. Brown and J. A. Cooper, Biochem. Biophys. Acta, 1996, 1287: 121-149; C. A. Lowell and P. Soriano, Genes Dev. 1996, 10: 1845-1857; S. M. Thomas and J. S. Brugge, Annu. Rev. Cell Dev. Biol. 1997, 13: 513-609). Kinases of the SRC family are implicated in cancer, immune system dysfunction and bone remodeling diseases (for a general review, see, for example, S. M. Thomas and J. S. Brugge, Annu. Rev. Cell Dev. Biol. 1997, 13: 513-609; and D. S. Lawrence and J. Niu, Pharmacol. Ther. 1998, 77: 81-114).

Based on published studies, SRC kinases are considered as important therapeutic targets for various human diseases. For example, Src has been reported as a particularly useful therapeutic target for bone diseases (P. Soriano et al., Cell, 1991, 64: 693-702), rheumatoid arthritis, for cancer such as colon, breast, hepatic and pancreatic cancer, certain B-cell leukemias and lymphomas (M. S. Talamonti et al., J. Clin. Invest. 1993, 91: 53-60; M. P. Lutz et al., Biochem. Biophys. Res. 1998, 243: 503-508; N. Rosen et al., J. Biol. Chem. 1986, 261: 13754-13759; J. B. Bolen et al., Proc. Natl. Acad. Sci. USA, 1987, 84: 2251-2255; T. Masaki et al., Hepatology, 1998, 27: 1257-1264; J. S. Biscardi et al., Adv. Cancer Res. 1999, 76: 61-119; S. A. Lynch et al., Leukemia, 1993, 7: 1416-1422; J. R. Wiener et al., Clin. Cancer Res. 1999, 5: 2164-2170; and C. A. Staley et al., Cell Growth Diff. 1997, 8: 269-274), as well as to develop inhibitors of the replication of hepatitis B virus (N. B. Klein et al., EMBO J. 1999, 18: 5019-5027, and N. B. Klein and R. J. Schneider, Mol. Cell. Biol. 1997, 17: 6427-6436). Other SRC family kinases are also potential therapeutic targets. These include, for example, Lck, which is well known as a therapeutic target for autoimmune diseases such as rheumatoid arthritis (T. J. Molina et al., Nature, 1992, 357: 161-164); and Hck, Fgr and Lyn, which have been reported as potential therapeutic targets for inflammation diseases (C. A. Lowell and G. Berton, J. Leukoc. Biol., 1999, 65: 313-320).

The screening methods of the invention may alternatively be used for identifying modulators of the phosphorylating activity of members of the JNK (jun-c kinase) family. Three distinct genes, JNK1, JNK2 and JNK3 have been characterized for this kinase family and at least ten different splicing isoforms of JNKs exist in mammalian cells (S. Gupta et al., EMBO J. 1996, 15: 2760-2770). Members of the JNK family are activated by pro-inflammatory cytokines, such as tumor necrosis factor-α (TNF-α) and interleukin-1β (IL-1β), as well as by environmental stress, including anisomycin, UV radiation, hypoxia, and osmotic shock (A. Minden and M. Karin, Biochem. Biophys. Acta, 1997, 1333: F85-F104). JNKs, along with other members of the MAP family, have a role in mediating cellular response to cancer, thrombin-induced platelet aggregation, immunodeficiency disorders, autoimmune disorders, cell death, allergies, osteoporosis and heart disease. The therapeutic targets related to activation of the JNK pathway include chronic myelogenous leukemia (CML) (G. M. Burgess et al., Blood, 1998, 92: 2450-2460), rheumatoid arthritis, asthma, hepatic ischemia (A. Behren et al., Nat. Genet. 1999, 21: 326-329; I. Onishi et al., FEBS Lett. 1997, 420: 201-204; M. Parola et al., J. Clin. Invest. 1998, 102: 1942-1950; and R. M. Zwacka et al., Hepatology, 1998, 28: 1022-1030), cancer (X. Xu et al., Oncogene, 1996, 13: 135-142), neurodegenerative diseases (A. A. Mohit et al., Neuron. 1995, 14: 67-78; D. D. Yang et al., Nature, 1997, 389: 865-870), and pathologic immune responses (S. Kempiak et al., J. Immunol. 1999, 162: 3176-3187; G. A. vanSeventer et al., Eur. J. Immunol. 1998, 28: 3867-3877; B. Dubois et al., J. Exp. Med. 1997, 186: 941-953; D. J. Wilson et al., Eur. J. Immunol. 1996, 26: 989-994).

Uses of Modulators of Kinase Activity

In another aspect, the present invention is directed to methods of using modulators of kinase activity. More specifically, a method is provided for inhibiting or enhancing a cellular biological response, wherein the biological response is associated or suspected to be associated with a disease or clinical condition, and wherein the biological response is mediated by events triggered by the phosphorylation of a substrate molecule inside a cell. The method includes contacting the cell with an effective amount of an inventive modulator of kinase activity.

A modulator of kinase activity according to the present invention may be administered to a cell in vitro, ex vivo or in vivo. In certain embodiments, the modulator of kinase activity is used to reduce/suppress the phosphorylating activity of a kinase inside a cell, thereby inhibiting the corresponding biological response(s) of the cell. Alternatively, the modulator is used to increase/enhance the phosphorylating activity of a kinase inside a cell, thereby stimulating the corresponding biological response(s) of the cell.

A modulator of kinase activity according to the present invention may, alternatively, be used in a system, such as a biological fluid, a biological tissue, or an animal (for example, an animal model for a particular human disease or clinical condition associated with cellular events triggered by the phosphorylation of a substrate molecule by a given kinase). For example, a modulator of kinase activity may be administered to the animal model in order to determine the efficacy, toxicity and side effects of a treatment with such a modulating agent; to elucidate the mechanism of action of such an agent, and/or to prevent or treat a disease or clinical condition affecting the animal.

Pharmaceutical Compositions

Modulators of the invention may be administered per se or in the form of a pharmaceutical composition. Accordingly, the present invention provides pharmaceutical compositions comprising at least one physiologically acceptable carrier and an effective amount of at least one modulator of kinase activity. The specific formulation of the modulator of kinase activity will depend upon the route of administration selected. Modulators, or pharmaceutical compositions thereof, may be administered by any suitable method known in the art. Examples of suitable routes include oral and parenteral administrations, including intravenous, intramuscular, intraperitoneal, and subcutaneous injections, transdermal and enteral administrations, and the like.

Dosage, mode of administration and formulation of a modulator of kinase activity (or pharmaceutical composition thereof) will depend on various parameters including the nature of the system (cell, biological fluid, biological tissue, or mammal) receiving the agent, the particular kinase activity to be altered or modulated, or the particular disease or physiological condition affecting the system.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### IL-2 Stimulated HT-2 Cell Signaling Assay

HT-2 is a murine helper-T cell line that is dependent on the cytokine, Interleukin 2 (IL-2), for its viability and proliferation. HT-2 cells die in the absence of IL-2 in the culture medium. The IL-2 receptor comprises a α chain, β chain, and γ chain. The γ chain binds to Janus kinase 3 (JAK3) while the α-chain binds to Janus kinase 1 (JAK1).

Ligand-induced oligomerization of the IL-2 receptor brings the receptor-associated JAKs into close proximity, which leads to auto-phosphorylation and activation of JAK3. Activated JAK3 phosphorylates the receptor chains and JAK1. This causes latent cytoplasmic STAT (Signal Transducer and Activator of Transcription) proteins to bind to the activated receptor complex. JAK3 then phosphorylates tyrosine residues of these receptor-bound STAT proteins. Phosphorylated STATs dimerize and translocate to the nucleus of the cell, where they bind to STAT binding elements on the promoters of STAT responsive genes thereby triggering transcription.

The cell-based assay described below allows identification of candidate compounds exhibiting the ability to modulate the tyrosine kinase activity of JAK3, when the kinase is stimulated by IL-2 inside a HT-2 cell. The method includes determination of the amount of tyrosine phosphorylated STAT-5 using a Guava 96-PCA well plate reader (Guava Technologies (Hayward, Calif.)).

Cell Culture

HT-2 clone A5E cells were obtained from the American Type Culture Collection (ATCC, Manassas, Va.; Cat # CRL-1841).

The cells were maintained in the following medium: RPMI 1640 (JRH Biosciences), 2 mM L-glutamine adjusted to contain 1.5 g/L sodium bicarbonate, 4.5 g/L glucose, 10 mM HEPES, 1.0 mM sodium pyruvate, 0.05 mM 2-mercaptoethanol, fetal bovine serum (10%), Rat T-STIM factor (Fisher Scientific) with Con A (10% by volume). The cultures were maintained by addition or replacement of fresh medium, and sub-cultured every two to three days at 3-5×10⁴ viable cells/mL.

Cell Starvation

HT-2 cells were counted, washed and resuspended at a density of 5×10⁶ cells per mL of fresh starving medium (i.e., same medium as culture medium described above except that the starving medium did not contain Rat T-STIM). The cells were starved for 4 hours at 37° C. in a humidified incubator. Following the starvation period, 50 μL (0.25×10⁶ cells) of the cell suspension were plated per well of a 96-V-bottom-well assay plate (Corning-Costart).

Candidate Compound Preparation

Candidate compounds to be tested were diluted in DMSO in a 96-well plate (in order to obtain concentrations of 10, 3.3, 1.11, 0.37, 0.123, 0.04, 0.0137 and 0.00046 mM). 2 μL of these dilutions were added to 500 μL of medium in 96-well cluster tubes so that the final concentration in medium was 2×. The resulting solutions were well mixed by pipeting up and down 4 to 5 times.

Cell Incubation in the Presence of Compounds to be Tested

For each candidate compound, 100 μL of the previous dilutions were added in triplicate to wells containing cells in suspension. 100 μL of medium plus DMSO were then added to each well, and the plate was kept in a humidified 37° C. incubator for 1 hour. Control cells were incubated under similar conditions except for the presence of a candidate compound.

IL-2 Stimulation and Plate Preparation

After incubation in the presence (or absence) of candidate compounds to be tested, 50 μL of recombinant murine IL-2 (R & D systems, Inc.) at 40 ng/mL (4×) were added per well while 50 μL of medium were added to the no IL-2 control cells. The plates were then incubated at 37° C. for 15 minutes.

Following IL-2 stimulation, the plates were centrifuged at 1000 rpm for 5 minutes. The supernatant was then removed by aspiration and 50 μL of 3.7% formaldehyde were added in each well to fix the cells (for each plate, a solution containing 0.5 mL 37% formaldehyde (Sigma) and 4.5 mL of 1×PBS (JRH Biosciences) was prepared fresh for each experiment). The plates were incubated on a plate shaker for 5 minutes at room temperature. They were then centrifuged at 1000 rpm for 5 minutes. The supernatants were removed by aspiration and 50 μL of 90% methanol (JT Baker) were added to each well to permeabilize the cells. The plates were incubated on ice for 30 minutes. At this time, if desired, the assay can be stopped and the plates can be stored at −20° C. for up to one month before being analyzed.

Staining and Analysis

At the time of analysis, the plates were centrifuged, the supernatants were removed by aspiration and the cells were washed with PBS.

25 μL of 1:10 diluted PS-5 PE antibody (Phospho STAT-5 (Y694) PE conjugate; BD Biosciences/Pharmingen, San Diego, Calif.) were then added per well. The plates were incubated for 45 minutes at room temperature on a plate shaker. Then 100 μL of PBS were added to each well and the plates were centrifuged. The supernatants were removed by aspiration and the cells of each well were resuspended in 100 μL of PBS. Each plate was then analyzed using the Guava PCA-96 plate reader.

FIGS. 1 and 2 show the results obtained for a candidate compound tested according to this inventive kinase assay.

### Example 2

### GM-CSF Stimulated TF-1 Cell Signaling Assay

TF-1 is an erythroleukemia cell line that is dependent on the growth factor GM-CSF (Granulocyte Macrophage-Colony Stimulating Factor) for growth. GM-CSF is a member of the gp 140 family of cytokines (which also comprises IL-3 and IL-5).

The common β chain cytoplasmic domain of the GM-CSF receptor is associated with Janus kinase 2 (JAK2). Cytokine stimulation induces heterodimerization with the α chain, which activates JAK2. Activated JAK2 then phosphorylates the receptor chains and STAT5 is recruited from the cytoplasm and binds to the activated receptor complex. STAT5 is then phosphorylated at tyrosine residues by JAK2. On phosphorylation, STAT5 dimerizes and translocates to the cell nucleus where it binds to STAT binding elements on promoters of STAT response genes, thus leading to transcription.

The cell-based assay described below allows identification of candidate compounds with the ability to modulate the tyrosine kinase activity of JAK2, when JAK2 is stimulated by GM-CSF in a TF-1 cell. The method includes determination of the amount of tyrosine-phosphorylated STAT-5 using the Guava 96 well plate reader.

Cell Culture

TF-1 cells were obtained from ATCC (Cat. # CRL-2003). The cells were maintained in the following medium: RPMI 1640 (JRH Biosciences), 2 mM L-glutamine adjusted to contain 1.5 g/L sodium bicarbonate, 4.5 g/L glucose, 10 mM HEPES, 1.0 mM sodium pyruvate, fetal bovine serum (10%), recombinant human GM-CSF (rhGMCSF; R & D systems, Inc.) (2 ng/mL). The cultures were maintained by addition or replacement of fresh medium. Usually cultures were started using 2×10⁵ cells/mL and maintained between 2×10⁵ and 1×10⁶ cells/mL.

Cell Starvation

TF-1 cells were counted, washed and resuspended at a density of 5×10⁶ cells per mL of fresh starving medium (same as culture medium described above except that the starving medium did not contain rhGM-CSF). The cells were starved for 4 hours at 37° C. in an incubator. Following the starvation period, 50 μL (0.25×10⁶ cells) of the cell suspension were plated per well in a 96-V bottom well assay plate (Corning-Costart).

Candidate Compound Preparation

Compounds to be tested were diluted in DMSO in a 96-well plate (in order to obtain concentrations of 10, 3.3, 1.11, 0.37, 0.123, 0.04, 0.0137 and 0.00046 mM). 2 μL of these dilutions were added to 500 μL of medium in 96-well cluster tubes so that the final concentration in medium was 2×. The resulting solutions were well mixed by pipeting up and down 4 to 5 times.

Cell Incubation in the Presence of Compounds to be Tested

For each compound to be tested, 100 μL of the previous dilutions were added in triplicate to wells containing cells in suspension. 100 μL of medium plus DMSO were then added to each well, and the plate was kept in a humidified 37° C. incubator for 1 hour. Control cells were incubated under the same conditions and for the same time in the absence of a candidate compound.

GM-CSF Stimulation and Plate Preparation

After incubation in the presence (or absence) of compounds to be tested, 50 μL of rhGMCSF (R & D systems, Inc.) at 10 ng/mL (4×) were added per well while 50 μL of medium were added to the no rhGMCSF control cells. The plates were then incubated at 37° C. for 15 minutes.

Following centrifugation of the plates at 1000 rpm for 5 minutes, the supernatant was removed by aspiration and 50 μL of 3.7% formaldehyde were added in each well to fix the cells (for each plate a solution containing 0.5 mL 37% formaldehyde (Sigma) and 4.5 mL 1×PBS (JRH Biosciences) was prepared fresh for each experiment). The plates were incubated on a plate shaker for 5 minutes at room temperature, and then centrifuged at 1000 rpm for 5 minutes. The supernatants were removed by aspiration and 50 μL of 90% methanol (JT Baker) were added to each well to permeabilize the cells. The plates were incubated on ice for 30 minutes. At this point, if desired, the assay can be stopped and the plates can be stored at −20° C. for up to one month before being analyzed.

Staining and Analysis

At the time of analysis, the plates were centrifuged; the supernatants were removed by aspiration; and the cells were washed with PBS.

25 μL of 1:10 diluted PS-5 PE antibody (Phospho STAT-5 (Y694) PE conjugate; BD Biosciences/Pharmingen) were then added per well. The plates were incubated for 45 minutes at room temperature on a plate shaker. Then 100 μL of PBS were added to each well and the plates were centrifuged. The supernatants were removed by aspiration and the cells of each well were resuspended in 100 μL of PBS. Each plate was then analyzed using the Guava PCA-96 plate reader.

FIGS. 3 and 4 show the results obtained in the case of a candidate compound tested according to this inventive kinase assay.

1. A method for measuring the phosphorylating activity of an enzyme in cells, wherein the enzyme is a kinase catalyzing the phosphorylation of a substrate molecule, the method comprising:
providing cells in a plurality of wells of a multi-well assay plate;
exposing the cells to a fluorescently-detectable selective probe such that the probe binds to the phosphorylated substrate;
measuring the amount of probe bound to the phosphorylated substrate using a Flow Cytometry Plate Reader, wherein the amount of bound probe is proportional to the amount of phosphorylated substrate; and
based on the amount of bound probe measured, determining the phosphorylating activity of the kinase.

2-4. (canceled)

5. The method of claim 1, wherein the multi-well assay plate is a 42-well plate, 96-well plate, 384-well plate or 1536-well plate.

6. The method of claim 5, wherein the multi-well assay plate is a 96-well plate and wherein between about 1×10⁴ and about 50×10⁴ cells are comprised in each one of the plurality of wells containing cells.

7-11. (canceled)

12. The method of claim 1, wherein the kinase is constitutively active.

13. The method of claim 1, wherein the kinase is non-constitutively active and the method further comprises starving the cells and then exposing the cells to a kinase activator such that activation of the kinase takes place and results in phosphorylation of the substrate prior to exposing the cells to the fluorescently-detectable selective probe.

14. (canceled)

15. The method of claim 13, wherein the kinase activator is selected from the group consisting of an environmental stress signal, a chemical stress signal, a biochemical stimulus, and any combination thereof.

16. The method of claim 15, wherein the kinase activator is a) an environmental stress signal selected from the group consisting of osmotic shock, heat shock, hypoxia, and UV radiation; b) is a chemical stress signal selected from the group consisting of hydrogen peroxide, diamine, sodium arsenite, cadmium chloride, and mercury chloride; c) a biochemical stimulus selected from the group consisting of a growth factor, a cytokine, a growth hormone, and a neurotransmitter; d) a growth factor selected from the group consisting of EGFs, FGFs, CSFs, HGFs, IGFs, ILGFs, NGFs, PDGFs, and VEGFs; or e) a cytokine selected from the group consisting of interleukins, interferons, and tumor necrosis factors.

17. The method of claim 1, wherein the substrate molecule is a downstream protein kinase, a gene regulatory protein, a cytoskeletal protein or a metabolic enzyme.

18-19. (canceled)

20. The method of claim 1, wherein exposing the cells to a fluorescently-detectable selective probe comprises adding to the cells a phospho-specific antibody comprising a fluorescent label, wherein the phospho-specific antibody recognizes and binds to at least one phosphorylated residue of the phosphorylated substrate.

21. The method of claim 1, wherein exposing the cells to a fluorescently-detectable selective probe comprises:
adding a phospho-specific antibody to the cells, wherein the phospho-specific antibody recognizes and binds to at least one phosphorylated residue of the phosphorylated substrate; and
adding to the cells a secondary antibody comprising a fluorescent label, wherein the secondary antibody specifically binds to the phospho-specific antibody.

22-29. (canceled)

30. A method for identifying a compound that modulates the phosphorylating activity of an enzyme in cells, wherein the enzyme is a kinase catalyzing the phosphorylation of a substrate molecule, the method comprising:
providing cells in a plurality of wells of a multi-well assay plate;
incubating cells in some wells of the assay plate with a candidate compound under conditions and for a time sufficient to allow equilibration, thus obtaining test cells;
incubating cells in other wells of the assay plate under the same conditions and for the same time absent the candidate compound, thus obtaining control cells;
exposing the test and control cells to a fluorescently-detectable selective probe such that the selective probe binds to the phosphorylated substrate;
measuring the amount of selective probe bound to the phosphorylated substrate in the test and control cells using a Flow Cytometry Plate Reader, wherein the amount of selective probe is proportional to the amount of phosphorylated substrate;
comparing the amount of bound probe in the test and control cells, and
determining that the candidate compound modulates the phosphorylating activity of the kinase if the amount of bound probe in the test cells is less than or greater than the amount of bound probe in the control cells.

31. The method of claim 30, wherein said method is used to identify a candidate compound that inhibits the phosphorylating activity of the kinase.

32-35. (canceled)

36. The method of claim 30, wherein the multi-well assay plate is a 42-well plate, 96-well plate, 384-well plate or 1536-well plate.

37. The method of claim 36, wherein the multi-well assay plate is a 96-well plate and wherein between about 1×10⁴ and about 50×10⁴ cells are comprised in each one of the plurality of wells containing cells.

38-42. (canceled)

43. The method of claim 30, wherein the kinase is constitutively active.

44. (canceled)

45. The method of claim 30, wherein incubating cells with the candidate compound comprises adding the candidate compound to a well containing cells.

46. The method of claim 45, wherein the candidate compound is added at a final concentration of between about 10 pM and about 100 μM.

47-49. (canceled)

50. The method of claim 30, wherein the kinase is non-constitutively active and wherein the method further comprises, prior to exposing the test and control cells to a fluorescently-detectable selective probe, exposing the test and control cells to a kinase activator such that activation of the kinase takes place and results in phosphorylation of the substrate.

51. The method of claim 50, wherein the kinase activator is selected from the group consisting of an environmental stress signal, a chemical stress signal, a biochemical stimulus, and combinations thereof.

52. The method of claim 50, wherein the kinase activator is a) an environmental stress signal selected from the group consisting of osmotic shock, heat shock, hypoxia, and UV radiation; b) a chemical stress signal selected from the group consisting of hydrogen peroxide, diamine, sodium arsenite, cadmium chloride, and mercury chloride; c) a biochemical stimulus selected from the group consisting of a growth factor, a cytokine, a growth hormone, and a neurotransmitter; d) a growth factor selected from the group consisting of EGFs, FGFs, CSFs, HGFs, IGFs, ILGFs, NGFs, PDGFs, and VEGFs; or e) a cytokine selected from the group consisting of interleukins, interferons, and tumor necrosis factors.

53. The method of claim 30, wherein the substrate molecule is a downstream protein kinase, a gene regulatory protein, a cytoskeletal protein or a metabolic enzyme.

54. The method of claim 30, further comprising:
fixing the test and control cells; and
permeabilizing the test and control cells that have been fixed, prior to exposing the test and control cells to the fluorescently-detectable selective probe.

55. (canceled)

56. The method of claim 30, wherein exposing the test and control cells to a fluorescently-detectable selective probe comprises adding to the test and control cells a phospho-specific antibody comprising a fluorescent label, wherein the phospho-specific antibody recognizes and binds to at least one phosphorylated residue of the phosphorylated substrate.

57. The method of claim 30, wherein exposing the test and control cells to a fluorescently-detectable selective probe comprises:
adding to the test and control cells a phospho-specific antibody, wherein the phospho-specific antibody recognizes and binds to at least one phosphorylated residue of the phosphorylated substrate; and
adding to the test and control cells a secondary antibody comprising a fluorescent label, wherein the secondary antibody specifically binds to the phospho-specific antibody.

58-65. (canceled)

66. The method of claim 30, wherein the candidate compound is incubated at different concentrations in different wells containing cells.

67. The method of claim 66, further comprising determining the IC₅₀ value of the candidate compound.

68. The method of claim 30, further comprising using a positive or negative control compound.

69. The method of claim 68, further comprising comparing the modulating effects of the candidate compound to the modulating effects of the positive or negative control compound.
